SEQUENCE LISTING (7 sequences)

SEQ ID NO: 1 - DNA, 29 bp, Artificial Sequence
  synthetic primer
gactgctggaaggacacatcgaagctgtg

SEQ ID NO: 2 - DNA, 29 bp, Artificial Sequence
  synthetic primer
gtgataggatcttctacatactgggcatg

SEQ ID NO: 3 - protein, 17 aa, human
CNYEDDLLGAAKPRYFY

SEQ ID NO: 4 - DNA, 20 bp, Artificial Sequence
  synthetic primer
tgggcggcatggtgcaagtt

SEQ ID NO: 5 - DNA, 20 bp, Artificial Sequence
  synthetic primer
cggtgctaaccagcgttttc

SEQ ID NO: 6 - DNA, 21 bp, Artificial Sequence
  synthetic primer
agtcttcagggatgacacaga

SEQ ID NO: 7 - DNA, 21 bp, Artificial Sequence
  synthetic primer
tacgaaactagagcacagcta

(19) United States
(12) Patent Application Publication
(10) Pub. No.: US 2006/0286090 A1
(43) Pub. Date: Dec. 21, 2006

(54) Mediators of epithelial adhesion and their role in cancer and skin disorders

(76) Inventors: Laura D. Attardi, Palo Alto, CA (US); Rebecca Ann Ihrie, Pacifica, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/408,306

(22) Filed: Apr. 21, 2006

Related U.S. Application Data
(60) Provisional application No. 60/674,154, filed on Apr. 21, 2005.

(51) Int. Cl. A61K 39/395; A01K 67/027; C12Q 1/68; G01N 33/574

(52) U.S. Cl. 424/131.1; 435/7.23; 435/6; 800/3

(57) ABSTRACT

Perp is shown to mediate stratified epithelial development in vivo. Perp localizes specifically to desmosomes, adhesion junctions important for tissue integrity. In some embodiments of the invention, detection of autoantibodies specific for perp find use in the diagnosis of pemphigus. In other embodiments, agent that upregulate or mimic perp function are useful in treatment of such blistering diseases. In another embodiment, agents that inhibit perp function are useful in treating and preventing cancer.

### INTRODUCTION

This invention was made with Government support under contract CA93665-03 awarded by the National Cancer Institute. The Government has certain rights in this invention.

The best characterized of the stratified epithelia is the epidermis of the skin, a self-renewing organ that regenerates approximately every 2 weeks. In skin, the basal cells of the innermost layer of the epidermis constitute the proliferative compartment renewing the epidermis. Progenitor cells of this basal layer, called transit amplifying cells, divide several times before withdrawing from the cell cycle, detaching from the basement membrane, and embarking on a migration and differentiation program. This differentiation process culminates with the formation of a keratinized cell layer, the stratum corneum, which provides a protective surface that also serves a barrier function.

Among the specializations most critical for stratified epithelial formation are cell-cell adhesive complexes including adherens junctions, tight junctions and desmosomes. These intercellular junctions promote interaction between epithelial cells, facilitating the mechanical linkage of cells or barrier function. Desmosomes are specialized adhesive junctions that are particularly important for the structural integrity of tissues subject to mechanical stress, such as the skin or the oral epithelia. Desmosomes assemble when the transmembrane desmosomal cadherins interact to bring the plasma membranes of adjacent cells in close apposition and nucleate cytoplasmic complexes of plakoglobin, plakophilin and desmoplakin to form a structure called the desmosomal plaque. Through linkage of the plaque to the keratin intermediate filament cytoskeleton via desmoplakin, desmosomes allow the formation of a supracellular network that endows an epithelium with strength and resiliency. The crucial role of desmosomal proteins in tissue integrity is underscored by the various human blistering diseases resulting from defects in desmosomal function.

Pemphigus is a group of rare autoimmune blistering diseases of the skin and/or mucous membranes. The disease is characterized by autoantibodies specific for desmosomes components. Often these antibodies react with desmogleins, which are important in maintaining the structural integrity of the skin by attaching adjacent cells. When autoantibodies attack desmogleins, the cells become separated from each other. The skin virtually becomes unglued. This causes burn-like lesions or blisters that do not heal. In some cases, these blisters can cover a significant area of the skin.

There are several types of Pemphigus, and early diagnosis is important. Criteria for diagnosis include the clinical presentation of skin lesions, lesion biopsy, and immunodiagnostic assays for the presence of autoantibodies. For patients with localized disease, topical steroids or intralesional steroids may be used. For patients with more severe or widespread disease, systemic corticosteroids, such as prednisone, are commonly used. Several other drugs are often used in combination with prednisone.

The p53 tumor suppressor is the most commonly mutated gene in human cancer, with over half of all human cancers sustaining p53 mutations. Moreover, p53 null mice are highly tumor-prone, succumbing universally to cancer within 10 months after birth. Together, these findings underscore the crucial role p53 plays in preventing cancer. p53 suppresses tumorigenesis by responding to various cellular stresses, such as DNA damage, hypoxia, or hyperproliferation, and inducing cells to undergo cell cycle arrest or apoptosis. Activation of either the cell cycle arrest or apoptotic pathway provides a mechanism to limit the propagation of potentially oncogenic cells. p53 is a transcriptional activator that engages these pathways at least in part through the induction of target genes involved in cell cycle arrest or apoptosis. While a host of p53 target genes have been identified, the importance of most of these genes in tumor suppression in vivo has not been clearly elucidated.

p53 is a member of a multi-protein family of transcription factors, including the p63 and p73 proteins. Whereas p53's main function is in tumor suppression, p63 and p73 play more A critical developmental roles, as deduced from the analysis of mice lacking these gene products. p73 is essential for neurogenesis and pheromone sensing, and p63 is required for the development of a variety of stratified epithelia, including the skin, hair follicles and oral mucosa. Although these proteins have important developmental functions, their role in cancer has been controversial, as few cases of mutations in either p63 or p73 in cancers have been reported. However, some studies indicate that there is interplay between p53 and its family members, suggesting that in some contexts, p73 or p63 might also participate in restricting tumorigenesis. For example, mice expressing p53 tumor mutants have a dominant predisposition to developing metastatic epithelial cancers that are thought to result from the binding and neutralization of p73 and p63 by mutant p53. Similarly, mice with compound mutations in p53 and p63, or p53 and p73, develop tumor types not observed in the single mutants, indicating cooperativity between these mutant alleles. These findings suggest that p63 and p73 provide back-up systems that limit tumorigenesis in the absence of p53. Target genes regulated by more than one p53 family member therefore may be very important for tumor suppression.

The Perp gene was identified in a screen for p53 target genes selectively induced during p53-mediated apoptosis rather than during cell cycle arrest. Perp overexpression is sufficient to induce cell death, and certain cell types lacking Perp display compromised apoptotic responses upon treatment with DNA damage. Together, these findings indicate that Perp is an important component of the p53 apoptotic pathway.

The genesis of a specialized tissue from a simple germ layer relies on the implementation of elaborate transcriptional programs. p63, a member of a family of transcription factors including the p53 tumor suppressor, plays a vital role in the development of stratified epithelia. Mice lacking p63 display post-natal lethality with a dramatic absence of limbs and skin due to the failure of these epithelia to develop during gestation. Ectopic expression of p63 in simple epithelia in vivo is sufficient to initiate critical aspects of stratification, suggesting that p63 performs an instructive role in this developmental program. Although these findings underscore the essential nature of p63 function in the determination of stratified epithelia, the specific pathways by which p63 acts are unknown.

How p63 might implement sub-programs fundamental to stratified epithelial development and integrity, including adhesion, proliferation, and polarity, is unknown. p63 may transactivate a cohort of genes that determine the identity and structure of stratified epithelia, but to date, p63 target genes clearly involved in stratified epithelial function in vivo have not been identified. Determination of such genes and their role in normal and pathological processes is of great scientific and clinical interest.

### SUMMARY OF THE INVENTION

Perp, a tetraspan membrane protein, is shown to mediate stratified epithelial development in vivo. Perp localizes specifically to desmosomes, adhesion junctions important for tissue integrity, and numerous structural defects in desmosomes are observed in Perp-deficient skin, demonstrating a role for Perp in promoting the stable assembly of desmosomal adhesive complexes. These findings demonstrate that Perp is a key effector in the p63 developmental program, playing an essential role in an adhesion sub-program central to epithelial integrity and homeostasis.

In some embodiments of the invention, detection of autoantibodies specific for perp find use in the diagnosis of pemphigus. In other embodiments, agent that upregulate or mimic perp function are useful in treatment of such blistering diseases.

In another embodiment, agents that modulate perp function are useful in treating and preventing cancer. Cancers associated with overexpression of Perp relative to normal tissue include, without limitation, squamous cell carcinoma of the lung and skin; hepatocellular carcinoma, renal cell carcinoma, and breast carcinoma. Perp is also associated with skin cancers, e.g. melanoma, basal cell carcinoma, etc.

It is shown that in the early stages of cancer development, e.g. in skin cancer, inhibition of perp can prevent the development of initial lesions. Other effects may be operable in later stages of cancer. Animals deficient in Perp in the skin are resistant to papilloma development, displaying fewer and smaller papillomas than wild-type controls. Proliferation levels, apoptotic indices and differentiation patterns are similar in the skin of treated Perp-deficient and wild-type animals.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1I. Perp Expression During Development. (A) and (B) Perp expression in early embryogenesis (E9.5-10.5) is observed in the apical ectodermal ridge (AER) of the developing limb buds (red arrows), and the branchial arches (blue arrows) by whole mount in situ hybridization. Digoxigenin-labeled probe localization appears as dark purple precipitate. (C-E) This pattern of expression is absent in p63−/− embryos (arrows in C, E), but is present in p53−/−embryos (arrows in D). (F) Detection of Perp mRNA is specific, as staining is absent in a Perp null embryo. (G) At E16.5, Perp mRNA is localized to many epithelia throughout the embryo by ³⁵S in situ hybridization. Radioactive probe localization appears as white precipitate. Perp is highly expressed in the skin (arrow), tongue (arrow), palate, and hair follicles. Additionally, Perp mRNA was detected in other tissues, including the heart (H), forestomach (F), submandibular gland (SMG), thymus (T), and bladder (B). (H) Perp protein is expressed in stratified but not simple epithelia in the adult mouse, as determined by Western blot analysis. GAPDH serves as a loading control. (I) Perp protein is observed during stratified epithelial development by immunohistochemistry. Skin sections of embryos from E11.5 to E18.5 are shown, with Perp immunohistochemistry marked by brown precipitate at the right of each panel, and accompanying H & E staining at the left.

FIG. 2A-2G. Perp is a p63 Target Gene. (A) Northern blot analysis shows robust Perp expression in wild-type and p53−/−, but not p63−/−, embryonic carcass tissue at E14.5. GAPDH serves as a loading control. (B) p63 is necessary for Perp expression in primary mouse keratinocytes. Infection of keratinocytes with retroviruses that do not affect p63 levels (either a GFP-expressing construct or a construct expressing an ineffective short hairpin RNA [shRNA] directed against p63, “p63 hairpin 2”) has no effect on Perp message levels, as determined by Northern blot. In contrast, expression of an shRNA that severely diminishes p63 mRNA levels (“p63 hairpin 1”) sharply reduces Perp message levels. (C) Western blot analysis shows that expression of p63 hairpin 1 efficiently reduces p63 protein levels. (D) A variety of p63 isoforms are capable of transactivating the Perp reporter construct (PerpLuc-wt), consisting of the promoter and part of the first intron fused to luciferase, in luciferase assays performed in p53−/−;p63−/− MEFs. Mutation of the intronic p53/p63 binding site in the Perp first intron (site D) compromises this transactivation activity (PerpLuc-mut). The graph shows the fold activation of the reporter by the expressed proteins relative to the empty vector, and represents the average of three experiments □SEM. (E) All p63 isoforms transactivate the wild-type Perp luciferase reporter construct in primary human keratinocytes. The average of four experiments □SEM is shown. (F) Chromatin immunoprecipitation on newborn mouse skin shows that p63 binds the p53/p63 consensus site in the Perp first intron (site D). Ethidium-stained agarose gels show a PCR product encompassing site D upon immunoprecipitation with p63 antibodies but not in a mock immunoprecipitation without chromatin or an immunoprecipitation using an isotype control nonspecific antibody. In addition, no PCR product was observed in the p63 chromatin immunoprecipitation using irrelevant primers for a portion of the Perp gene lacking a p53 binding site. (G) Northern blot analysis demonstrates,that Perp mRNA is expressed at low levels in cultured E18.5 p63−/− ectoderm, but is efficiently induced upon infection with a TAp6□-expressing adenovirus

FIG. 3A-3L. Perp−/− Mice Exhibit Post-Natal Lethality and Defects in Stratified Epithelia. (A) Targeting scheme for generating Perp null mice. A targeting construct in which Perp exons 2 and 3 were flanked by IoxP sites (triangles) was introduced into 129/Sv embryonic stem cells. These exons were then deleted via introduction of Cre recombinase, resulting in a null allele, and ES cells were used to generate Perp−/− mice. (B) Genotypes of 3-week old progeny derived from Perp heterozygote intercrosses. The numbers of wild-type, Perp± and Perp−/− mice observed on either a mixed 129/Sv-C57BL/6 or a pure 129/Sv genetic background are indicated. (C) Appearance of Perp null pups at P5. Whereas wild-type pups appear normal, Perp−/− pups show signs of wasting. (D) Survival of Perp null newborns after birth. Kaplan-Meier analysis demonstrates that whereas most wild-type and Perp± mice survive until weaning, the vast majority of Perp−/− mice die within the first week of life. (E) Wild-type oral mucosa (P, palate and T, tongue) develops normally at P7.5. (F) In contrast, Perp−/− animals exhibit severe lesions in the oral cavity, primarily blisters between the basal and immediate suprabasal layers of the epithelium (arrows). (G) Higher magnification view of a basal-suprabasal blister in a Perp −/− tongue. (H) Many Perp−/− mice exhibit multiple lesions (arrows) as well as debris in the throat. (I-L) The skin of P7.5 Perp−/— mice (J, L) is also aberrant when compared to wild-type littermates (I, K). Perp−/− epidermis is thicker than that of wild-type mice (brackets in K and L) and also exhibits blistering (J, arrow).

FIG. 4A-4G. Perp Protein Localizes to the Desmosome. (A) and (B) Indirect immunofluorescence on P0.5 wild-type skin with anti-Perp antibodies reveals a punctate pattern of staining at the cell membrane. In (B), DAPI staining indicates the basal (nucleated) layer of the epidermis. Dashed lines indicate the basement membrane of the epidermis. (C) This staining is absent in Perp−/− mouse skin. (D-F) Immunogold EM using anti-Perp antibodies shows that Perp localizes specifically to desmosomes in P0.5 wild-type mouse skin (D and E), but not in Perp−/− mouse skin (F). (G) Perp staining is absent from other regions of cell-cell contact (blue arrows), confirming specific localization to desmosomes.

FIG. 5A-5G. Epithelia From Perp−/− Mice Exhibit Aberrant Desmosomes. (A) Wild-type mouse skin contains many desmosomes that are identifiable by EM (red arrows) and show connections to the intermediate filaments (blue arrow). (B, C, E) By contrast, desmosomes are less frequent in Perp−/− mouse skin and also exhibit other abnormalities, including decreased electron density (red arrows, B), lack of attachment to intermediate filaments (blue arrow in B), and evident lack of mechanical integrity demonstrated by their detachment from one of the apposed cells (arrows, C and E). (D) Normal desmosomes are also present in the wild-type tongue. (F) Desmosome abnormalities are also present in Perp−/− mouse tongue (arrow) when compared to wild-type tongue. (G) The properties of desmosomes in wild-type and Perp−/− mouse skin are quantified in the table shown. In total, a high percentage of desmosomes in Perp−/− mouse skin were scored as abnormal based on lack of connection to keratin filaments, decreased electron density, or loss of adhesive function.

FIG. 6A-6P. Desmoplakin Localization is Abnormal in Perp−/− Keratinocytes. (A-J) Examination of desmosomal protein localization in keratinocytes derived from wild-type and Perp−/− mouse skin. (A, B) Adherens junction formation (marked by E-cadherin localization) is unaltered in Perp−/− keratinocytes. (C, D) Similarly, desmoglein 3 localizes normally in Perp−/− keratinocytes. (E, F) Localization of plakoglobin appears normal in Perp−/− keratinocytes. (G, H) Keratin intermediate filaments appear, normal in Perp−/− keratinocytes when compared to wild-type cells. (I, J) Localization of the desmosomal plaque protein desmoplakin (DP) is significantly altered in Perp−/− cells. Whereas DP typically localizes to the plasma membrane in wild-type keratinocytes (I), a significant fraction of DP in Perp−/− cells remains in the cytoplasm (J). (K,L) This difference is evident widely in the population through a low power view. (M, N) This difference may also be detected in Perp−/− skin, where a greater fraction of DP is distributed within the cells (N) rather than localizing to the membrane as in wild-type skin (M). (O, P) Perp−/− skin also exhibits abnormal keratin 14 aggregation (P) when compared to wild-type skin (O).

FIG. 7A-7D. Desmosomal Protein Solubility is Perturbed in Perp−/− Mouse Skin. (A) Western blot analysis demonstrates that desmosomal proteins are present at higher levels in total protein extracts from Perp−/− keratinocytes compared to wild-type keratinocytes, whereas non-desmosomal proteins are unaffected. K14 serves as a loading control. (B) Enhanced Triton X-100 solubility is observed for the desmosomal proteins desmoglein 1, desmoglein 3 and plakoglobin in fractions derived from Perp−/− keratinocytes and analyzed by Western blot. E-cadherin and claudin 1 show similar solubility in wild-type and Perp−/− keratinocytes. GAPDH and K14 serve as loading controls for the Triton X-100-soluble and -insoluble fractions, respectively. (C) Desmosomal proteins are present at approximately equal levels in wild-type and Perp−/− total mouse skin, as determined by Western blot analysis. The adherens junction protein E-cadherin and the tight junction protein claudin 1 are also unaffected. (D) Triton X-100 solubility profiles of desmosomal proteins in wild-type and Perp−/− mouse skin. Desmogleins 1 and 3 and plakoglobin are enriched in the Triton X-100 soluble fraction from Perp−/− skin. This effect is specific to desmosomal constituents, as E-cadherin and claudin 1 are unaffected.

FIG. 8A-8D. K5-Cre transgenic system to inactivate Perp in the skin. (A) Targeting scheme used to generate conditional (floxed) Perp M,ice. The generation of ES cells with the targeted 3-lox Perp allele has been described. Mice carrying the 3-lox Perp allele were bred to CMV-Cre transgenic mice (34) to obtain Perp conditional mice. (B) Western blot analysis to examine Perp levels in stratified epithelia (tongue, skin and esophagus) from K5-Cre transgenic; Perp fl/fl and control non-transgenic; Perp fl/fl mice. “fl” denotes the floxed allele. GAPDH serves as a loading control. (C-D) Immunohistochemistry to examine Perp protein levels and localization in K5-Cre Perp+/+ and K5-Cre; Perp fl/fl mice. For clarity, epidermis from DMBA/TPA-treated mice was examined, and hence appears thickened relative to untreated adult skin.

FIG. 9A-9D. Perp fl/fl mice are resistant to papilloma development. (A) Graph showing the average numbers and sizes of papillomas >2mm in K5-Cre; Perp +/+ mice as a function of time of TPA treatment. Color scale indicates the sizes of papillomas. (B) Graph showing the average numbers and sizes of papillomas >2mm in K5-Cre; Perp fl/fl mice as a function of time of TPA treatment. Color scale indicates the sizes of papillomas. (C) Representative images of K5-Cre; Perp+/+ and K5-Cre; Perp fl/fl mice showing the dramatic difference in papilloma numbers and sizes seen in the absence of Perp at week 28. (D) Graph showing the average number of papillomas greater than 2mm, ±standard error of the mean (SEM), at the time of death (between weeks 24 and 28), in K5-Cre transgenic mice that are either Perp+/+, Perp fl/+, or Perp fl/fl.

FIG. 10A-10B. Papilloma morphology is similar in K5-Cre; Perp+/+and K5-Cre; Perp fl/fl mice. (A) Hematoxylin and eosin staining of a portion of a typical papilloma in a K5-Cre; Perp +/+ mouse. (B) Hematoxylin and eosin staining of typical papilloma in a K5-Cre; Perp wn mouse.

FIG. 11A-11G. Proliferation and apoptosis levels are comparable in the epidermis of treated K5-Cre; Perp+/+ and K5-Cre; Perp fl/fl mice. (A, B) Proliferation index was determined by Ki67 staining of skin from K5-Cre; Perp+/+ and K5-Cre; Perp fl/fl mice after 27 weeks of TPA treatment. (C, D) DAPI labels all nuclei of the epidermis. (E) Graph showing the average percentage of basal cells of the epidermis displaying Ki67 positivity, ±SEM, in treated K5-Cre; Perp+/+ and K5-Cre; Perp fl/fl mice. (F, G) Apoptosis was assessed through TUNEL staining of skin from K5-Cre; Perp+/+ and K5-Cre; Perp fl/fl mice after 27 weeks of TPA treatment. Arrows indicate TUNEL-positive cells.

FIG. 12A-12F. The epidermal differentiation program occurs normally in the absence of Perp. Immunohistochemical analysis was used to examine the terminal differentiation program in the skin of K5-Cre; Perp+/+ and K5-Cre; Perp fl/fl mice treated for 27 weeks with TPA. (A, B) Keratin 14, typically a marker of the basal cell population in the skin, shows widespread expression in treated skin of mice of both genotypes. (C, D) Keratin 1 is a marker of the spinous layer of the skin. (E, F) Loricrin is a marker of, the granular layer of the epidermis.

FIG. 13A-13H. Desmosome function is perturbed in the skin of K5-Cre; Perp fl/fl mice. (A) Hematoxylin and eosin staining of newborn K5-Cre; Perp+/+ skin. (B) Hematoxylin and eosin staining of newborn K5-Cre; Perp fl/fl skin. A blister formed by separation of the basal and suprabasal layers of the epidermis is indicated by the arrow. (C) Hematoxylin and eosin staining of K5-Cre; Perp+/+ mouse skin treated with TPA for 27 weeks. (D) Hematoxylin and eosin staining of K5-Cre; Perp fl/fl mouse skin treated with TPA for 27 weeks. Note spaces between epithelial cells (arrows). (E) Higher magnification view of treated K5-Cre; Perp+/+ mouse skin. (F) Higher magnification view of treated K5-Cre; Perp fl/fl mouse skin showing separation between epidermal cells. (G) Western blot analysis examining Triton X-100 solubility profiles of desmosomal proteins in K5-Cre; Perp fl/fl and control (K5-Cre; Perp+/+ and Perp fl/fl) mouse skin. Tubulin and K14 serve as loading controls for the Triton X-100-soluble and -insoluble fractions, respectively. (H) Western blot analysis showing that total protein levels of desmoglein 1 and plakoglobin are similar regardless of genotype.

### DETAILED DESCRIPTION OF THE EMBODIMENTS

Methods and compositions are provided for the treatment and diagnosis of certain skin conditions involving the Perp protein. Perp is a tetraspan membrane protein, shown to mediate stratified epithelial development in vivo. Perp localizes specifically to desmosomes, adhesion junctions important for tissue integrity. In some embodiments of the invention, detection of autoantibodies specific for perp find use in the diagnosis of pemphigus.

Screening methods for therapeutic agents that modulate perp activity are also provided. In such methods, candidate compounds are tested for the ability to enhance or inhibit perp function. Such screening methods may include assays of biological function, e.g. the localization of Perp to desmosomes and the integrity of skin tissue. Such agents find use in the treatment of blistering diseases and of cancers associated with Perp, as described herein. For example, Perp inhibitors are useful in the prevention of cancer, e.g. skin cancer, and may be applied, topically or systemically, prior to exposure to carcinogens, or immediately following such exposure, e.g. in the event of radiation damage.

To that end any agent(s) that modulates the activity of Perp can be used as a therapeutic or prophylactic agent. The agent that acts to increase such activity can be a purified form of the gene product, an agent that stimulates expression or synthesis of the gene product, or a nucleic acid that includes a segment encoding a perp gene, or any agent that acts as a direct or indirect activator of the protein, e.g. a pharmacological agonist or antagonist.

Several characteristics of Perp (p53 effector related to PMP-22) make it a compelling candidate for such a target. Perp is a p53 target gene involved in DNA damage-induced apoptosis, and during this process, the Perp promoter is bound not only by p53 but also by p63, indicating that Perp is responsive to signals from p63. Moreover, Perp is unique among p53 targets because it encodes a tetraspan membrane protein, implicating it in cell-cell interactions. It is distantly related to members of the claudin/PMP-22/EMP family of four-pass membrane proteins. This multi-protein family includes stargazin, claudins, and PMP-22, which participate in a variety of cellular processes including ion channel function, receptor trafficking, tight junction formation and myelination. As a plasma membrane protein, Perp can act in a manner similar to any of these proteins to affect events important for tissue development or architecture.

Perp is useful in diagnostic assays for the detection of certain cancers, in which Perp is upregulated in comparison to surrounding normal; tissues. Cancers associated with overexpression of Perp relative to normal tissue include, without limitation, squamous cell carcinoma of the lung and skin; hepatocellular carcinoma, renal cell carcinoma, and breast carcinoma. Perp is also associated with skin cancers, e.g. melanoma, basal cell carcinoma, etc.

The genetic sequence of Perp has been described by Attardi et al. (2000) Genes and. Dev. 14:704-718, and in co-pending U.S. patent application Ser. No. 09/634,907, both of which are herein specifically incorporated by reference for the teaching of polynucleotide and polypeptide sequences, fragments and variants thereof.

### Disease Conditions for Diagnosis and Treatment

Pemphigus usually occurs in middle-aged or elderly persons and is rare in children. In active pemphigus, the serum and skin contain readily demonstrable IgG antibodies that bind at the site of epidermal damage. These antibodies can induce the same pathologic process in vivo and in vitro. The primary lesions are flaccid bullae of various sizes, but often the skin or mucosae just shear off, leaving painful erosions. Lesions typically occur first in the mouth, where they rupture and remain as chronic, often painful, erosions for variable periods before the skin is affected. The bullae typically arise from healthy-appearing skin, rupture, and leave a raw area and crusting. Any area of stratified squamous epithelium may be affected, but the extent of skin and mucosal involvement varies (eg, lesions may occur in the oropharynx and upper esophagus). Itching is usually absent.

Pemphigus is a serious disease with an inconsistent and unpredictable response to therapy, a prolonged course, and virtually inevitable complicating drug side effects. Specific therapy depends on the extent and severity of disease. The mainstay is systemic corticosteroids. Some patients with few lesions may respond to low-dose oral prednisone (eg, 20 to 30 mg/day), but most require much higher doses. Methotrexate, cyclophosphamide, azathioprine, gold, or cyclosporine used alone or with corticosteroids reduces the need for corticosteroids and thus minimizes the undesirable effects of long-term corticosteroid use, but the aforementioned drugs also carry serious risks. Plasmapheresis combined with an immunosuppressive drug to reduce antibody titers has also been effective. Active skin infections are treated with systemic antibiotics.

In pemphigus vulgaris, the most common form of pemphigus, there are IgG antibodies that bind to the cell surfaces of epidermis of the skin as well as the epithelium lining mucosal surfaces such as the mouth. As a result, patients develop severe oral ulcerations, and may also have inflammation or erosions of the lining of the eye and eyelids (conjunctiva), the nasal mucosa, or the genital mucosa. Half of the patients also develop blisters or erosions of the skin, often in the head and neck area.

In pemphigus foliaceus there are IgG antibodies that bind to the cell surfaces of epidermis of the skin. As a result, patients develop superficial cutaneous erosions. Paraneoplastic pemphigus is a recently-described autoimmune blistering disease that occurs in patients with certain types of cancers. This disorder, which is very rare, is characterized by severe ulceration of the mouth and lips, ulceration and scarring of the lining of the eye and eyelids (conjunctiva), and skin lesions that may include blisters or violaceous plaques. As the antibodies also bind the airways, patients may develop a variety of signs of respiratory disease, which is usually rapidly fatal. Almost all patients have an underlying cancer, which includes non-Hodgkin's lymphoma, chronic lymphocytic leukemia, Castleman's disease, thymoma, Waldenstrom's macroglobulinemia, and sarcomas. IgA pemphigus is a recently-described rare autoimmune blistering disorder in which IgA antibodies bind to the cell surface of epidermal cells. This disease is different from other types of pemphigus in that the type of antibodies are IgA instead of IgG. The disease may be similar to pemphigus foliaceus with erosions and some blisters or it may be characterized by numerous small pustules.

Prompt and sufficient doses of steroids, usually prednisone or prednisolone, are required to get pemphigoid under control. Once controlled,;the medications are reduced slowly to minimize side effects. A large number of patients experience remission; however, a maintenance dose is often required to keep the disease under control. Other drugs that find use include antibiotics and other immunosuppressants. Treatment is addressed according to the disease activity that is clinically apparent. An indirect immunofluorescence test (antibody titer count) will generally show a high count when the disease is more active, and will be low or undetectable when the disease is in remission.

Skin cancers, which are usually curable, are the most common type of cancer; most arise in sun-exposed areas of skin. The incidence is highest in outdoor workers, sportsmen, and sunbathers and is inversely related to the amount of melanin skin pigmentation; light-skinned persons are most susceptible. Skin cancers may also develop years after x-ray or radium burns or arsenic ingestion.

Basal cell carcinoma is the most common type of skin cancer, with >400,000 new cases yearly in the USA. It is more common in fair-skinned, sun-exposed persons and is very rare in blacks. The clinical presentation and biologic behavior of basal cell carcinomas are highly variable. They may appear as small, shiny, firm, almost translucent nodules; ulcerated, crusted papules or nodules; flat, scarlike indurated plaques; or red, marginated, thin papules or plaques difficult to differentiate from psoriasis or localized dermatitis. Most commonly the carcinoma begins as a shiny papule, enlarges slowly, and, after a few months or years, shows a shiny, pearly border with prominent engorged vessels (telangiectases) on the surface and a central dell or ulcer. Recurrent crusting or bleeding is not unusual, and the lesion continues to enlarge slowly. Basal cell carcinomas rarely metastasize but may invade healthy tissues.

Squamous cell carcinoma, the second most common type of skin cancer, may develop in normal tissue, in a preexisting actinic keratosis or patch of leukoplakia, or in burn scars. The incidence in the USA is 80,000 to 100,000 cases annually. The clinical appearance is highly variable. The tumor may begin as a red papule or plaque with a scaly or crusted surface and may become nodular, sometimes with a warty surface. In some, the bulk of the lesion may lie below the level of the surrounding skin. Eventually it ulcerates and invades the underlying tissue. The percentage of squamous cell carcinomas on sun-exposed skin that metastasize is quite low. However, about ⅓ of lingual or mucosal cancers have metastasized before diagnosis. Treatment is the same as for basal cell carcinoma, but treatment and follow-up must be monitored closely because of the greater risk of metastasis.

Malignant melanoma is a melanocytic tumor arising in a pigmented area: skin, mucous membranes, eyes, and CNS. About 25,000 new cases of malignant melanoma occur yearly in the USA, causing about 6000 deaths. The incidence-is rising rapidly. Sun exposure is a risk, as is family history and the occurrence of lentigo maligna, large congenital melanocytic nevus, and the dysplastic nevus syndrome. About 40 to 50% of malignant melanomas develop from pigmented moles; almost all the rest arise from melanocytes in normal skin. The very rare malignant melanomas of childhood almost always arise from large pigmented moles (giant congenital nevi) present at birth. Malignant melanomas vary in size, shape, and color (usually pigmented) and in their propensity to invade and metastasize. This neoplasm may spread rapidly, causing death within months of its recognition, yet the 5-yr cure rate of early, very superficial lesions is nearly 100%. Thus, cure depends on early diagnosis and early treatment.

Metastasis of melanoma occurs via lymphatics and blood vessels. Local metastasis results in formation of nearby satellite papules or nodules that may or may not be pigmented. Direct metastasis to skin or internal organs may occur, and occasionally metastatic nodules or enlarged lymph nodes are discovered before the primary lesion is identified. Melanomas arising from mucous membranes have a poor prognosis, although they often seem quite limited when discovered. Treatment is by surgical excision. Although the width of margins is debated, most experts agree that a 1-cm lateral tumor-free margin is adequate for lesions<1 mm thick. Thicker lesions may deserve more radical surgery and sentinel node biopsy.

Bronchogenic Carcinoma is a highly malignant lung tumor, which accounts for most cases of lung cancer. Cigarette smoking is the principal cause of bronchogenic carcinoma. A strong dose-response relationship occurs in the three most common types of bronchogenic carcinoma: squamous cell, small cell, and adenocarcinoma.

Four histologic types of bronchogenic carcinoma usually are distinguished: squamous cell, commonly arising in the larger bronchi and spreading by direct extension and lymph node metastasis; undifferentiated small cell, often associated with early hematogenous metastases; undifferentiated large cell, usually spreading through the bloodstream; and adenocarcinoma, commonly peripheral, often spreading through the bloodstream. All types also commonly spread via the lymphatics. Squamous cell carcinoma accounts for 25-30% of all lung cancers. The classic manifestation is a cavitary lesion in a proximal bronchus. This type is characterized histologically by the presence of keratin pearls and can be detected based on results from cytologic studies because it has a tendency to exfoliate. It is the type most often associated with hypercalcemia.

Manifestations depend on the tumor's location and type of spread. Because most bronchogenic carcinomas are endobronchial, patients typically present with cough, with or without hemoptysis. In patients with chronic bronchitis, increased intensity and intractability of preexisting cough suggest a neoplasm. Bronchial narrowing may cause air trapping with localized wheezing and commonly causes atelectasis with ipsilateral mediastinal shift, diminished expansion, dullness to percussion, and loss of breath sounds. Persistent localized chest pain suggests neoplastic invasion of the chest wall. Peripheral nodular tumors are asymptomatic until they invade the pleura or chest wall and cause pain or until they metastasize to distant organs. Late symptoms include fatigue, weakness, decreased activity, worsening cough, dyspnea, decreased appetite, weight loss, and pain.

Hepatocellular carcinoma is the most common internal malignancy and an important cause of death in certain areas of Africa and Southeast Asia. Chronic hepatitis B virus (HBV) infection is largely responsible for the high prevalence of the tumor in endemic areas; the risk is more than one hundredfold higher among HBV carriers, and tumor incidence generally parallels HBV prevalence geographically. More recently, chronic hepatitis C virus (HCV) infection has been recognized as an important factor in the genesis of hepatocellular carcinoma. Four major gross patterns and two special forms of HCC have been described. The first type is the expanding type, in which the tumor is encapsulated and grows by expanding, compressing, and distorting the surrounding parenchyma of the liver. Late in the course of the disease, satellite nodules or metastases are seen. The second pattern is the spreading type, in which the tumor is poorly defined and occurs in the setting of hepatic cirrhosis. The tumor growth pattern may be nodular, pseudolobular, or invasive. A third major type is a multifocal pattern in which several small tumors of similar size are found in multiple sites in the liver and in which a primary to secondary relationship is difficult, if not impossible, to determine. A combination or indeterminate pattern is seen in up to 25% of cases.

Histopathologically, grades of I to IV are assigned, according to the granularity and acidophilic quality of the cytoplasm, the size and degree of hyperchromatism of the nuclei, the proportion of the cell occupied by the nucleus, the cohesive quality of the tumor cells, and the overall architecture of the tumor. A grade IV hepatocellular carcinoma lacks cohesiveness of cells and shows enough cell spindling to resemble a carcinosarcoma, whereas grade I tumors mimic the normal liver parenchyma.

A number of immunohistochemical stains distinguish HCC from metastatic adenocarcinoma or cholangiocarcinoma. Antibodies to α-fetoprotein and α1-antitrypsin are of limited value because they lack sensitivity and specificity. Antibodies to the carcinoembryonic antigen (CEA) may produce a canalicular membrane-staining pattern in well-differentiated hepatomas, but will not produce the intracytoplasmic staining typical of metastatic adenocarcinoma and cholangiocarcinoma. Cytokeratin-19 antibodies stain adenocarcinomas but not neoplastic or normal hepatocytes. Erythropoiesis-associated antigen (Ery-1) is a specific marker for HCC.

Renal cell carcinoma accounts for about 2% of adult cancers. The male:female ratio is estimated at 3:2. Most solid kidney tumors are malignant. Gross or microscopic hematuria is the most common presenting sign, followed by flank pain, palpable mass, and FUO. Hypertension due to segmental ischemia or pedicle compression and polycythemia secondary to increased erythropoietin activity sometimes occur. Increasingly, renal cell carcinoma is incidentally detected by abdominal ultrasound (US) and CT. US or IVU confirms the presence of a mass, and CT offers information regarding its density, local extension, and nodal and, venous involvement. MRI offers further information regarding extension into adjacent structures, particularly the renal vein and vena cava, and has largely replaced inferior vena cavography. Metastatic renal cell carcinoma has a poor prognosis because it is radioresistant, and traditional chemotherapeutic drugs, alone or in combination, and progestational drugs are ineffective. In some patients, immunotherapy reduces tumor size and prolongs survival. Interleukin-2 has been approved for metastatic renal cell carcinoma; various combinations of this drug and other biologic agents are being investigated.

Cancers of interest also include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

### Diangostic, Prognostic, and Therapeutic Methods

The presence of antibodies specific for Perp in a sample can serve as markers for diagnosis, and in prognostic evaluations to detect individuals at risk for pemphigus disease. In general, such diagnostic and prognostic methods involve detecting an altered level of anti-Perp antibodies in the cells or tissue of an individual or a sample therefrom. A variety of different assays can be utilized to detect an increase in antibodies. More specifically, the diagnostic and prognostic methods disclosed herein involve obtaining a sample from an, individual and determining at least qualitatively, and preferably quantitatively, the level of specific antibodies in the sample. Usually this determined value or test value is compared against some type of reference or baseline value.

Samples can be obtained from a variety of sources. For example, since the methods are designed primarily to diagnosis and assess risk factors for humans, samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from whole blood, serum, semen, saliva, tears, urine, fecal material, sweat, buccal, skin, spinal fluid and amniotic fluid. Also included in the term are derivatives and fractions of such cells and fluids. Samples can also be derived from in vitro cell cultures, including the growth medium, recombinant cells and cell components. The number of cells in a sample will often be at least about 102, usually at least 103, and may be about 104 or more. The cells may be dissociated, in the case of solid tissues, or tissue sections may be analyzed. Alternatively a lysate of the cells may be prepared.

Diagnostic samples are collected any time after an individual is suspected to have disease lesions. In prophylactic testing, samples can be obtained from an individual who present with risk factors that indicate a susceptibility to disease.

The various test values determined for a sample from an individual believed to have pemphigus typically are compared against a baseline value to assess the extent of increased expression, if any. This baseline value can be any of a number of different values. In some instances, the baseline value is a value established in a trial using a healthy cell or tissue sample that is run in parallel with the test sample. Alternatively, the baseline value can be a statistical value (e.g., a mean or average) established from a population of control cells or individuals. For example, the baseline value can be a value or range that is characteristic of a control individual or control population. For instance, the baseline value can be a statistical value or range that is reflective of expression levels for the general population.

The differential expression of Perp in cancers as described herein indicates that Perp can serve as a marker for diagnosis, for imaging, as well as for therapeutic applications. In general, such diagnostic methods involve detecting an elevated level of expression of Perp transcripts or protein in the cells or tissue of an individual or a sample therefrom. A variety of different assays can be utilized to detect an increase in gene expression, including both methods that detect gene transcript and protein levels. More specifically, the diagnostic and prognostic methods disclosed herein involve obtaining a sample from an individual and determining at least qualitatively, and preferably quantitatively, the level of Perp expression in the sample. Usually this determined value or test value is compared against some type of reference or baseline value.

Nucleic acids or binding members such as antibodies that are specific for Perp polypeptides are used to screen patient samples for increased expression of the mRNA or protein, or for the presence of amplified DNA in the cell. Samples can be obtained from a variety of sources. Samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained as described above, from the tissues or fluids of an individual, as well as from cell cultures or tissue homogenates.

Nucleic Acid Screening Methods

Some of the diagnostic and prognostic methods that involve the detection of a Perp transcript begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample.

A number of methods are available for analyzing nucleic acids for the presence of a specific sequence, e.g. upregulated expression. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp.14.2-14.33.

A detectable label may be included in an amplification reaction. Suitable labels include fluorochromes, e.g. ALEXA dyes (available from Molecular Probes, Inc.); fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. 32P, 35S, 3H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence.

Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g. the flat surface of a microscope slide or the walls of a microtiter well) and then permeabilizing the cells with an appropriate permeabilizing solution. A solution containing labeled probes is then contacted with the cells and the probes allowed to hybridize. Excess probe is digested, washed away and the amount of hybridized probe measured. This approach is described in greater detail by Nucleic Acid Hybridization: A Practical Approach (Hames, et al., eds., 1987).

A variety of so-called “real time amplification” methods or “real time quantitative PCR” methods can also be utilized to determine the quantity of mRNA present in a sample. Such methods involve measuring the amount of amplification product formed during an amplification process. Fluorogenic nuclease assays are one specific example of a real time quantitation method that can be used to detect and quantitate transcripts. In general such assays continuously measure PCR product accumulation using a dual-labeled fluorogenic oligonucleotide probe—an approach frequently referred to in the literature simply as the “TaqMan” method. Additional details regarding the theory and operation of fluorogenic methods for making real time determinations of the concentration of amplification products are described, for example, in U.S. Pat No. 5,210,015 to Gelfand, U.S. Pat. No. 5,538,848 to Livak, et al., and U.S. Pat. No. 5,863,736 to Haaland, each of which is incorporated by reference in its entirety.

Polypeptide Screening Methods

Screening for expression of Perp may be based on the functional or antigenic characteristics of the protein. Detection may utilize staining of cells or histological sections, performed in accordance with conventional methods, using antibodies or other specific binding members that specifically bind to Perp. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and Perp in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding. After incubation, the insoluble support is generally washed of non-bound components. After washing, a solution containing a second antibody is applied. The antibody will bind to one of the proteins of interest with sufficient specificity such that it can be distinguished from other components present. The second antibodies may be labeled to facilitate direct, or indirect quantification of binding. In a preferred embodiment, the, antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the targeted polypeptide, conveniently using a labeling method as described for the sandwich assay.

In some cases, a competitive assay will be used. In addition to the patient sample, a competitor to the targeted protein is added to the reaction mix. The competitor and the target compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

Imaging in vivo

In some embodiments, the methods are adapted for imaging use in vivo, e.g., to locate or identify sites where tumor cells are present. In these embodiments, a detectably-labeled moiety, e.g., an antibody, which is specific for Perp is administered to an individual (e.g., by injection), and labeled cells are located using standard imaging techniques, including, but not limited to, magnetic resonance imaging, computed tomography scanning, and the like.

For diagnostic in vivo imaging, the type of detection instrument available is a major factor in selecting a given radionuclide. The radionuclide chosen must have a type of decay that is detectable by a given type of instrument. In general, any conventional method for visualizing diagnostic imaging can be utilized in accordance with this invention. Another important factor in selecting a radionuclide for in vivo diagnosis is that its half-life be long enough that it is still detectable at the time of maximum uptake by the target tissue, but short enough that deleterious radiation of the host is minimized. A currently used method for labeling with ⁹⁹m Tc is the reduction of pertechnetate ion in the presence of a chelating precursor to form the labile ⁹⁹m Tc-precursor complex, which, in turn, reacts with the metal binding group of a bifunctionally modified chemotactic peptide to form a ⁹⁹m Tc-chemotactic peptide conjugate.

The detectably labeled Perp specific antibody is used in conjunction with imaging techniques, in order to analyze the expression of the target. In one embodiment, the imaging method is one of PET or SPECT, which are imaging techniques in which a radionuclide is synthetically or locally administered to a patient. The subsequent uptake of the radiotracer is measured over time and used to obtain information about the targeted tissue. Because of the high-energy (γ-ray) emissions of the specific isotopes employed and the sensitivity and sophistication of the instruments used to detect them, the two-dimensional distribution of radioactivity may be inferred from outside of the body.

Among the most commonly used positron-emitting nuclides in PET are included ¹¹C, ¹³N, ¹⁵O, and ¹⁸F. Isotopes that decay by electron capture and/or γ emission are used in SPECT, and include ¹²³I and ^{99m}Tc.

The detection methods can be provided as part of a kit. Thus, the invention further provides kits for detecting the presence of an anti-Perp antibody, or for Per quantitation, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

Agents that modulate activity of Perp provide a point of therapeutic or prophylactic intervention in pemphigus diseases and with respect to cancers as described herein. Numerous agents are useful in modulating this activity, including agents that directly modulate expression, e.g. expression vectors, antisense specific for the targeted protein; molecules that induce expression in the host cell, and agents that act on the protein, e.g. specific antibodies and analogs thereof, small organic molecules that block catalytic activity, etc.

Antisense or RNAi molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA, or RNAi molecules. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Compounds identified by the screening methods described herein and analogs thereof can serve as the active ingredient in pharmaceutical compositions formulated for the treatment of various skin disorders. The compositions can also include various other agents to enhance delivery and efficacy. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. The dosage of the active ingredient typically lies within a range of circulating concentrations that include the ED50 with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The components used to formulate,the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Compound Screening

Compound screening may be performed using an in vitro model, a genetically altered cell or animal, or purified perp protein. One can identify ligands or substrates that bind to, inhibit, modulate or mimic the action of the encoded polypeptide.

The polypeptides include those encoded by Perp, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by Perp, or a homolog thereof.

Transgenic animals or cells derived therefrom are also used in compound screening. Transgenic animals may be made through homologous recombination, where the normal locus corresponding to Perp is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in biological activity, oncogenesis, signal transduction, etc. Of interest is the use of Perp to construct transgenic animal models for skin conditions. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development. By providing expression of the target protein in cells in which it is not normally produced, one can induce changes in cell behavior.

Compound screening identifies agents that modulate function of perp. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of inhibiting or otherwise altering, or mimicking the physiological function of Perp. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to Perp, as at least some of the compounds so identified are likely inhibitors. The binding assays usually involve contacting perp with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The perp protein utilized in such assays can be naturally expressed, cloned or synthesized.

Cell based assays for perp function may involve contacting a cell population that is capable of being induced to form desmosomes with a candidate agent, and comparing the induced desmosomes and cells in the absence and presence of the compound. For example, keratinocytes, either in a primary culture or a cell line, can be induced to form desmosomes with a calcium switch. When desmosomes are not formed under these conditions, the cells comprise a variety of changes, including morphology, localization of proteins in the plasma membrane that would otherwise form desmosomes, response to stress, and the like.

Certain screening methods involve screening for a compound that modulates the expression of perp. Such methods generally involve conducting cell-based assays in which test compounds are contacted with one or more cells expressing perp and then detecting an increase in Perp expression (either transcript or translation product).

Gene expression can be detected in a number of different ways. The expression level of Perp in a cell can be determined by probing the mRNA expressed in a cell with a probe that specifically hybridizes with a transcript (or complementary nucleic acid derived therefrom) of Perp. Probing can be conducted by lysing the cells and conducting Northern blots or without lysing the cells using in situ-hybridization techniques (see above). Alternatively, Perp can be detected using immunological methods in which a cell lysate is probe with antibodies that specifically bind to Perp.

Other cell-based assays are reporter assays conducted with cells that do not express Perp. Certain of these assays are conducted with a heterologous nucleic acid construct that includes a perp promoter, which may include the regulatory element found in the first intron of the perp gene, that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase; Alton and Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression of Perp and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

The level of expression or activity can be compared to a baseline value. As indicated above, the baseline value can be a value for a control sample or a statistical value that is representative of expression levels for a control population (e.g., healthy individuals not at risk). Expression levels can also be determined for cells that do not express Perp as a negative control. Such cells generally are otherwise substantially genetically the same as the test cells.

Various controls can be conducted to ensure that an observed activity is authentic including running parallel reactions with cells that lack the reporter construct or by not contacting a cell harboring the reporter construct with test compound. Compounds can also be further validated as described below.

Compounds that are initially identified by any of the foregoing screening methods can be further tested to validate the apparent activity, e.g. to detect the activity of the compound in the treatment of pemphigus, squamous cell carcinoma, skin cancer, hepatocellular carcinoma, renal cell carcinoma, breast carcinoma, etc. The basic format of such methods involves administering a lead compound identified during an initial screen to an in vitro or in vivo model for the disease, and determining whether an effect is observed on the specific disease condition, e.g. inhibition of cancer cell growth, etc. The animal models utilized in validation studies generally are mammals. Specific examples of suitable animals include, but are not limited to, primates, mice, and rats.

Active test agents identified by the screening methods described herein can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate perp protein activity. Such compounds can then be subjected to further, analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Here, we demonstrate that Perp is a principal player in, the p63-directed program of stratified epithelial development. We show that Perp displays a striking epithelial-specific expression pattern during embryogenesis and that its expression depends on direct activation by p63. Through the characterization of Perp−/− mice, we determine that Perp has a pivotal role in cell-cell adhesion by enabling desmosome function. These studies identify Perp as the first direct molecular target of p63 definitively involved in stratified epithelial function in vivo. Our results provide an initial understanding of the sub-programs dictating specific tissue attributes downstream of p63, a global regulator of epithelial development, integrity and homeostasis.

Results

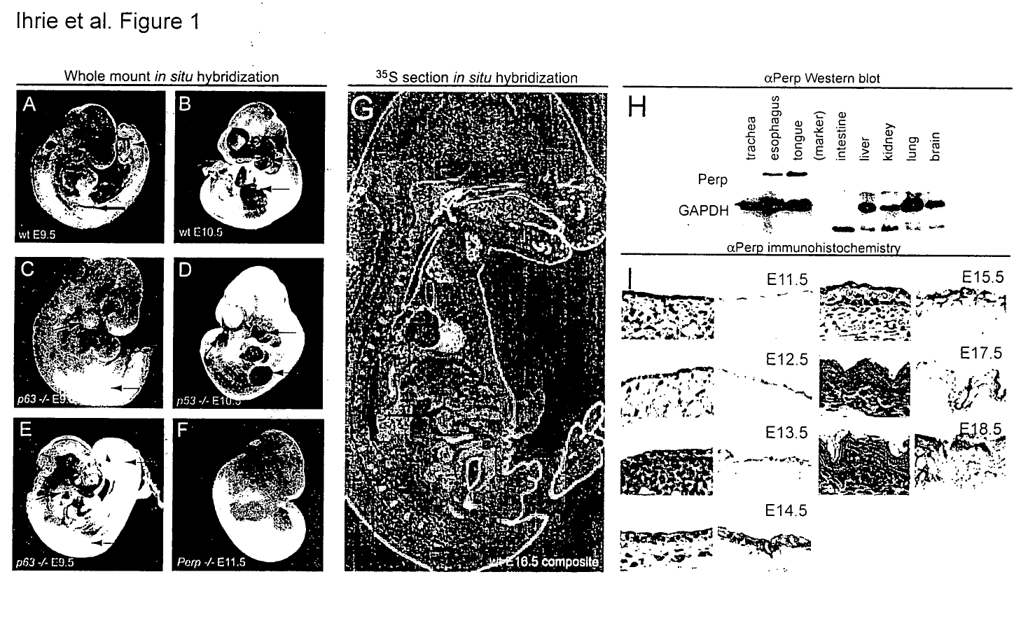

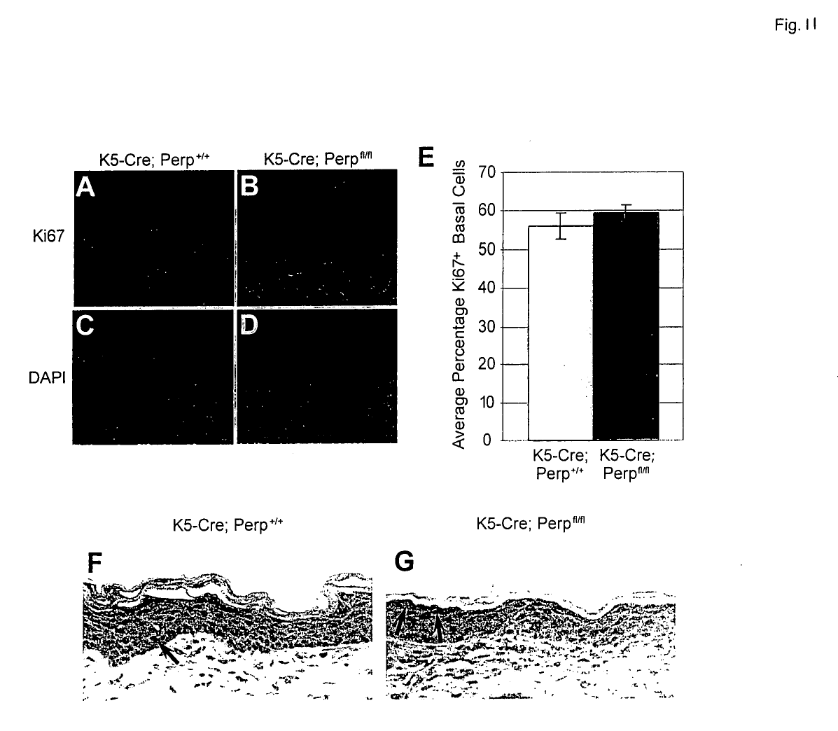

Perp is Highly Expressed in Stratified Epithelia. To elucidate the developmental role for Perp in vivo as a potential target of p63, we began by determining the spatial localization pattern of Perp mRNA during embryogenesis. In early embryos (E9.5-10.5), Perp mRNA expression was observed in the ectoderm of the developing branchial arches and limb buds, as well as in surface ectoderm, by whole mount in situ hybridization (FIG. 1A, 1B, data not shown). At later stages of development (E16.5-18.5), Perp mRNA localized predominantly to epithelia, most notably the oral mucosa and skin (FIG. 1G). Analysis of Perp protein levels in a variety of adult mouse tissues by Western blot and immunohistochemistry confirmed its expression in stratified epithelia, including the skin and tongue, but not in simple epithelia (FIG. 1H, S2H, data not shown). To determine the profile of Perp protein expression during the conversion of ectoderm to stratified epidermis, which occurs from approximately E9.5-E1 5.5, we performed immunohistochemical analysis (FIG. 11). We found Perp protein expressed in developing skin during and after the stratification process, consistent with a role for Perp in this compartment.

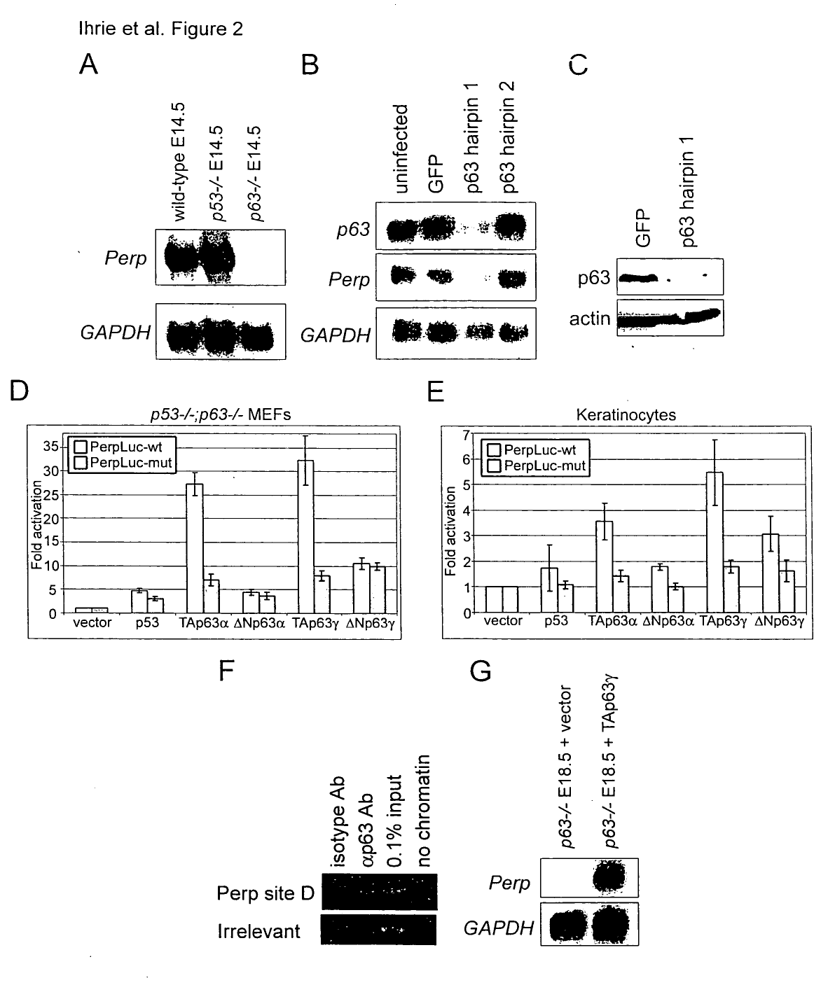

Perp is a p63 Target Gene. The pattern of Perp mRNA expression in the branchial arches and developing limb buds in a profile coincident with that observed for p63 mRNA, as well as its specific localization to stratified epithelia, suggested Perp might be a target of p63 during epithelial development (FIG. 1A, 1B, 1I). To test this hypothesis, we analyzed Perp mRNA expression in E9.5 p63−/− embryos, before the.7 defects associated with p63 loss compromised the integrity of the tissues that express Perp. We found that Perp mRNA was absent from the limb buds and branchial arches in p63−/− embryos (FIG. 1C, 1E), but was unaltered in p53‘/− embryos (FIG. 1D). Consistent with these results, Northern blot analysis demonstrated that Perp mRNA is present in both wild-type and p53−/−, but not p63−/−, E14.5 body tissue (FIG. 2A). The absence of Perp expression is specifically due to p63-deficiency and not a lack of the appropriate tissue specified by p63, as RNA interference experiments showed that ablation of p63 in primary mouse keratinocytes results in severely diminished Perp expression (FIG. 2B, 2C). Together, these findings indicate that Perp expression during embryogenesis requires the presence of p63, but not p53, and are consistent with the idea that Perp is a key target of p63 in maturing stratified epithelia.

To determine if p63 directly regulates Perp expression, we pursued several approaches. We first tested whether p63 could transactivate a reporter construct comprising the Perp promoter and a segment of the first intron fused to luciferase. To eliminate background effects of endogenous p63 or p53, we introduced p63 expression constructs into p63−/−;p53−/− MEFs along with the Perp reporter construct (FIG. 2D). p63 exists in multiple forms, including transactivating (TA) isoforms and ΔN isoforms lacking amino-terminal transactivation sequences, each transcribed from distinct promoters. Moreover, each of these isoforms can be alternatively spliced to yield the α, β, and γ variants.

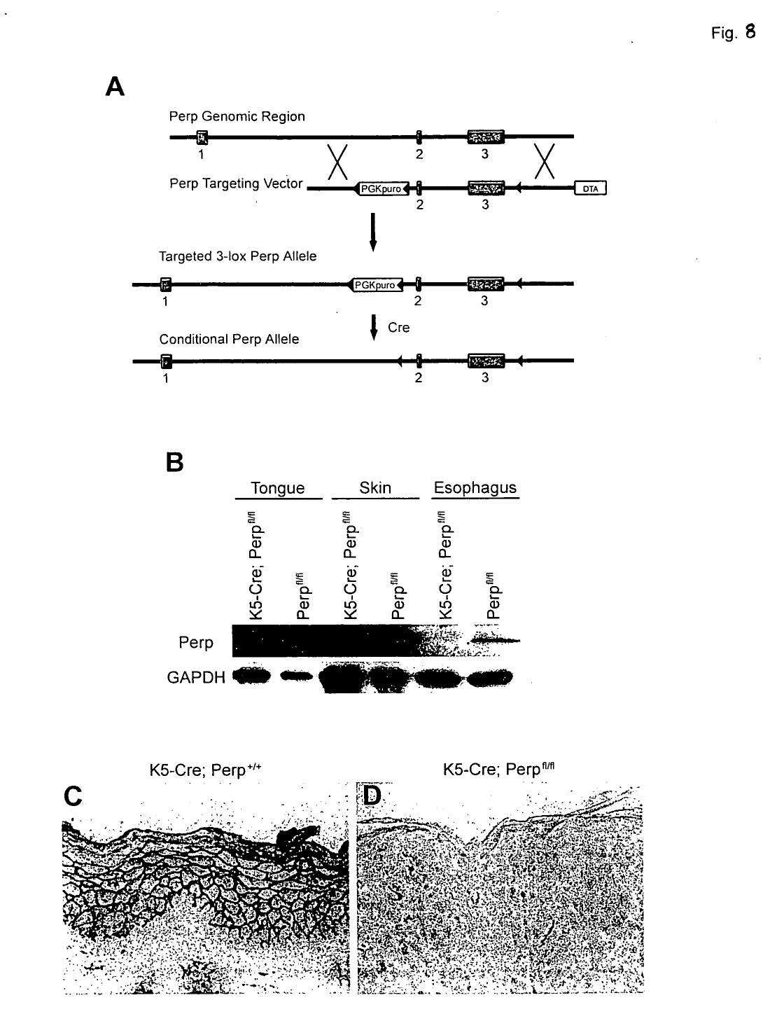

As the roles of each isoform in vivo may differ, we tested several isoforms in our assays. We found that TAp63α and TAp63γ both robustly transactivated the Perp reporter construct, and this was mediated largely through the p53/p63 consensus element in intron 1, the major p53-responsive site in Perp (FIG. 8 2D; Reczek et al., 2003). The .Np63 isoforms also transactivated the reporter efficiently, to levels ˜50% of those seen with TAp63 isoforms. The ability of p63 to induce the Perp reporter was confirmed through experiments in wild-type keratinocytes, which indicated that both TAp63 and □Np63 isoforms can directly activate Perp expression in cells where p63 exerts its function (FIG. 2E). To determine if p63 directly binds the Perp endogenous regulatory region in vivo, we performed chromatin immunoprecipitation (ChIP) assays (FIG. 2F). We found that p63 protein in newborn mouse skin binds the intron 1 p53/p63 site, the key p63-responsive element in the Perp regulatory region. Further support of the idea that p63 is a major regulator of Perp was provided by experiments showing that TAp6□expression is sufficient to drive endogenous Perp mRNA expression in cultured E18.5 p63‘/− ectodermal cells (FIG. 2G). Taken together, these results implicate Perp as a direct p63 target gene during skin development.

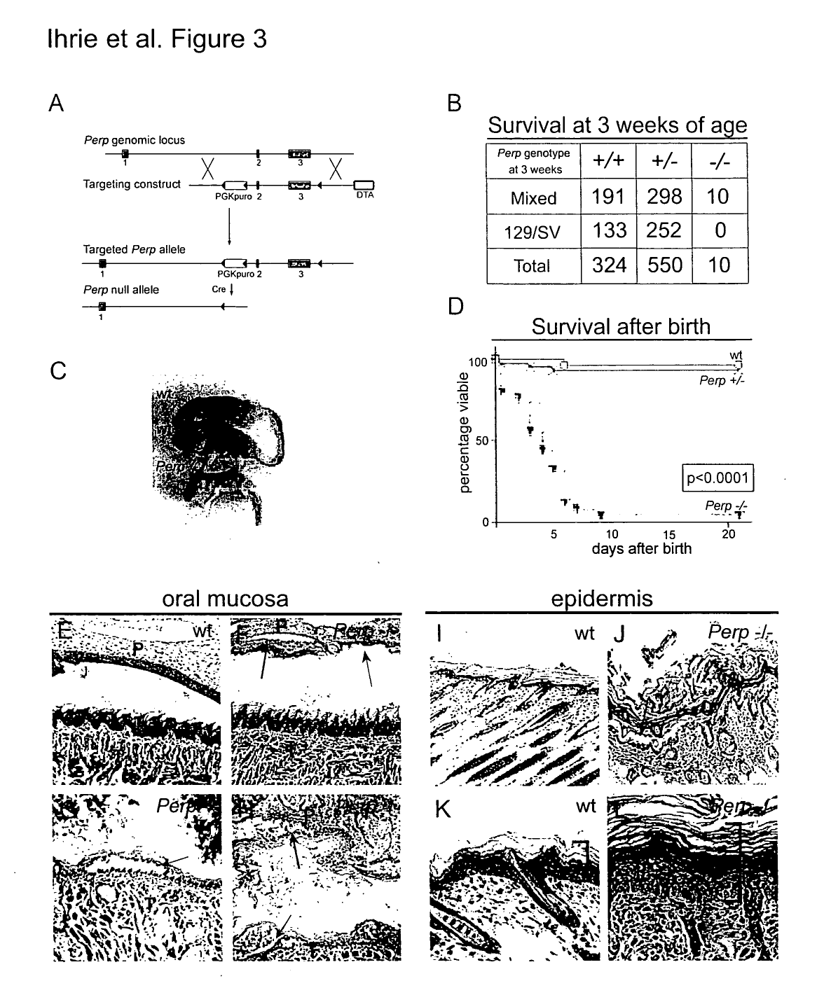

Perp is Essential for Post-Natal Viability. To determine the specific function Perp might perform in the p63 stratified epithelial development program in vivo, we generated Perp knockout mice. We deleted exons 2 and 3 of the Perp gene, which encode the majority of the protein, in ES cells, and used these targeted cells to generate Perp heterozygous mutant mice (FIG. 3A). Northern blot and immunohistochemistry experiments verified that this Perp allele is a null (FIG. S2H, S2I, Ihrie et al., 2003). Perp± mice were intercrossed, and the frequency of offspring of each genotype was examined at 3 weeks of age (FIG. 3B). We examined mice both on a mixed 129/Sv;C57BL/6 background and on a pure 129/Sv background. Whereas these crosses produced wild-type and heterozygous mice in abundance, homozygous mutant mice were drastically underrepresented at weaning, with no Perp null mice surviving on a pure 129/Sv background. An extremely limited number of mixed background Perp−/− mice survived until adulthood, but these mice ultimately also exhibited increased mortality (FIG. 3B).

To establish when Perp-deficiency compromises viability, we genotyped both embryos and neonatal progeny from Perp heterozygote intercrosses. We found that although Perp null mice survive until birth, the vast majority die within ten days after birth, with signs of wasting at the time of death (FIG. 3C, 3D). This lethal phenotype is in striking contrast to the viability of p53−/− mice, suggesting that the role of Perp in development is p53-independent, and raising the possibility that the requirement for Perp may reflect instead a specific role as a target of p63 in epithelia.

Perp−/− Mice Display Dramatic Blistering of the Skin and Oral Mucosa. The prominent stratified epithelial pattern of Perp expression and its position downstream of p63 suggested a potential role for Perp in these tissues. We therefore focused our initial pathological examination on these epithelial structures to discover the cause of mortality in Perp−/− newborn mice. Although the skin and oral mucosa of postnatal day 0.5 (P0.5) Perp−/− newborns appeared histologically similar to those of wild-type littermates (data not shown), examination of the same tissues in P7.5 Perp−/− mice revealed the presence of severe blisters (FIG. 3E-L). Analysis of the oral cavities of many Perp−/− mice (n=14) showed multiple blisters of the oral epithelia, as well as thickening of these tissues (FIG. 3F-H). The blisters most commonly observed were splits between the basal and suprabasal layers of cells, often with disrupted adhesion between individual cells, known as acantholysis (FIG. 3G). In addition to blistering in the oral mucosa, Perp−/− pups exhibited frequent basal-suprabasal blistering in the skin, as well as dramatic thickening of the epidermis (FIG. 3J, 3L). These phenotypes highlight a potential function for Perp in epithelial adhesion and provide a rationale for the lethality of Perp null newborns. The observed debris in the mouth and throat suggest that the mechanical stress of feeding progressively increases the severity of these blisters, ultimately impeding feeding and leading to the death of the mouse.

To determine if Perp−/− mice manifested any other phenotypes typical of p63 null mice, we examined tissues known to be affected by the absence of p63. p63−/− mice are born with craniofacial defects and truncated limbs, and lack ectodermal appendages such as teeth. In contrast, Perp−/− mice exhibited grossly normal craniofacial, limb and tooth development (FIG. S1A-D, data not shown). These findings suggest that Perp's requirement downstream of p63 is most prominent in tissues subject to significant mechanical stress, specifically the stratified epithelia of the skin and oral mucosa.

Apoptosis and Differentiation are Unaltered in Perp-Deficient Epithelia. Perp's function in epithelial integrity could reflect its known role in apoptosis or a novel, direct role in adhesion. We first examined apoptosis and proliferation in the Perp−/− newborn epithelia to determine if the observed blistering might be due to altered tissue homeostasis. Virtually no apoptotic cells were observed in either wild-type or Perp−/− skin using TUNEL staining (FIG. S2A, S2B). Perp−/− skin did exhibit increased staining for the proliferation marker Ki67 relative to wild-type skin in the basal layer of interfollicular epidermis (FIG. S2C, S2D, S2G), suggesting that the abnormal thickness of Perp−/− skin is directly due to hyperproliferation. Perp−/− skin also exhibited broad staining for keratin 6 (FIG. S2E, S2F), which is frequently induced in the interfollicular epidermis in response to adhesion defects, hyperproliferative signals, or wounding (Wong et al., 2000). This finding suggests that the enhanced proliferation observed in the Perp−/− mice could reflect a response to an adhesion defect rather than a cause. Despite this hyperproliferation, terminal differentiation markers appeared unaltered in Perp−/− skin when compared to wild-type skin, suggesting that neither the thickening nor the blistering phenotypes seen in Perp−/− skin are due to a differentiation defect (FIG. S2J-S2O). In light of these data, we designed experiments to elucidate what direct role Perp might play in cell-cell adhesion.

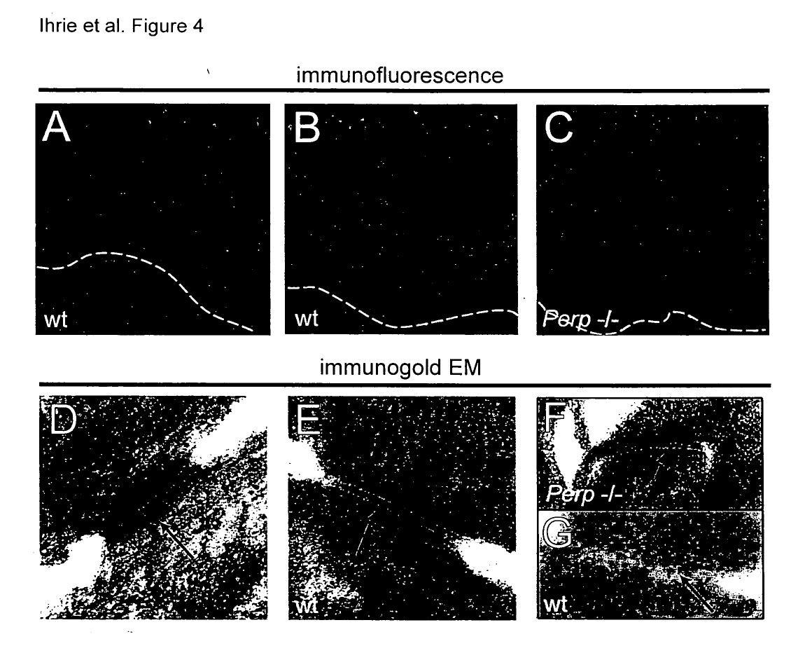

Perp Localizes to Desmosomes. To investigate the function of Perp in adhesion, we began by studying the subcellular localization of Perp in newborn skin. Indirect immunofluorescence using antibodies directed against Perp demonstrated that it is highly expressed in all layers of the skin. Closer examination of this staining pattern showed that Perp localizes to the plasma membrane and exhibits a punctate pattern similar to that observed with other adhesion proteins (FIG. 4A, 4B).

Proper cell-cell adhesion in the skin requires the presence of multiple adhesion complexes, including adherens junctions, desmosomes, and tight junctions (Fuchs and Raghavan, 2002). The punctate pattern of Perp staining suggested a potential association with one or more of these complexes. To distinguish these possibilities, we utilized Madin Darby canine kidney (MDCK) epithelial cells, a well-characterized model system for examining intercellular junctions in polarized epithelial layers (Yeaman et al., 2004). Cells were transfected with a construct encoding a Perp-GFP fusion protein before formation of a polarized confluent monolayer. By confocal microscopy, we found that overexpressed Perp-GFP did not co-localize with the tight junction protein ZO-1 (FIG. S3C, S3D) but showed some co-localization with both the desmosomal protein desmoplakin (FIG. S3A) and the adherens junction protein E-cadherin (FIG. S3B). To distinguish the two possible junctions that might contain Perp, we turned to immunogold electron microscopy (EM) on newborn skin, which allowed us to examine the precise localization of endogenous protein in vivo. The electron-dense structure of the desmosome is easily distinguishable by EM, and we found that the gold particles indicating Perp staining localized almost exclusively to the desmosome (FIG. 4D, 4E) and were entirely absent from other regions of cell-cell contact (FIG. 4G). These experiments demonstrate that endogenous Perp protein localizes specifically to the desmosome.

Desmosome Structure Is Perturbed in Perp Null Mice. The localization of Perp to the desmosome suggested that Perp may be important for function of the desmosome, which plays a central role in epithelial integrity and resiliency by linking cell-cell contact points to the keratin cytoskeleton and enhancing the ability of a tissue to resist stress. Consistent with this idea, mice deficient for other desmosomal components exhibit blistering symptoms histologically similar to those seen in Perp−/− mice. Moreover, the desmosomal cadherins desmoglein 1 and 3 are the primary antigens in the human autoimmune blistering diseases pemphigus foliaceus and pemphigus vulgaris, respectively, and patients with these diseases also develop blisters resembling those in Perp−/− mice. We therefore examined the desmosomes in the skin and tongue from Perp−/− newborns closely for structural abnormalities.

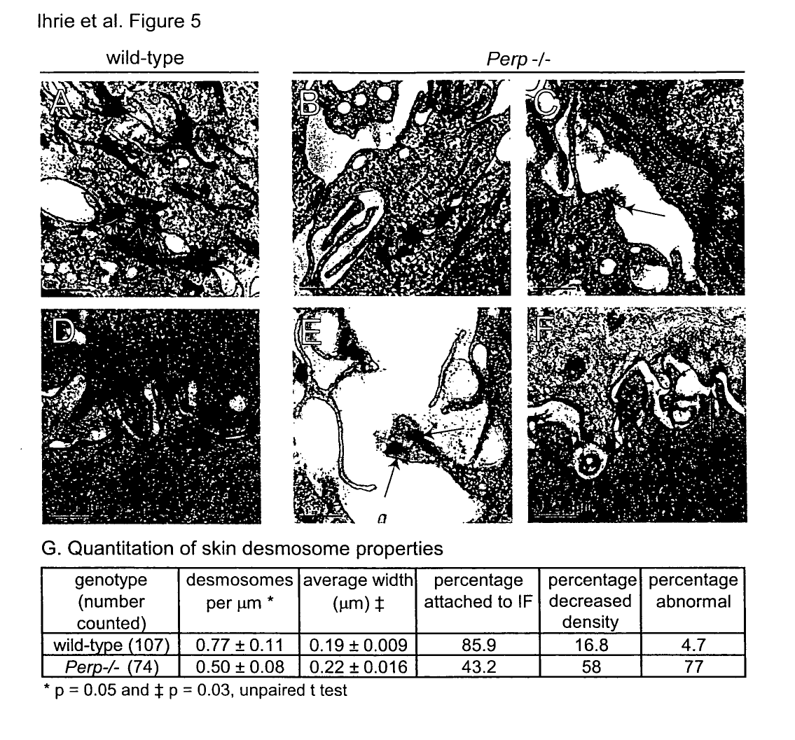

By EM, desmosomes are highly electron-dense structures, and typically appear as two adjacent dark plaques that partially or totally obscure the cellular membrane (red arrows, FIG. 5A). In addition, the connection of the desmosomal plaque to the keratin intermediate filaments, which are also electron-dense and therefore distinguishable by EM, often can be visualized. We focused our analysis on the basal and immediate suprabasal layers of skin, where the most severe blisters were evident in Perp−/− mice. At low magnification, some of the blisters present by histology were visible by EM (arrows, FIG. S4B, S4C). To assess the state of desmosomes in Perp−/− mouse skin, we scored them according to several morphometric criteria: 1) linear density (the number of desmosomes per μm of cellular membrane); 2) the average width of each desmosome, a property thought to reflect the assembly and organization of the desmosome; 3) the electron density of the desmosome relative to the adjoining plasma membrane, which likely indicates the protein content or assembly of the desmosome; 4) the attachment of the desmosome to intermediate filaments; and 5) any other abnormalities, such as obvious detachment from one of the two apposing plasma membranes.

Based on meeting these criteria, individual desmosomes were scored as normal or abnormal (FIG. 5G). Whereas desmosomes in wild-type skin appear very electron dense compared to the plasma membrane (red arrows, FIG. 5A), and frequently exhibit clear connections to the intermediate filaments (blue arrow, FIG. 5A), we found that desmosomes in Perp null.14 skin are typically abnormal. Desmosomes in Perp−/− skin are wider (FIG. 5G), less electron dense (FIG. 5B), rarely exhibit clear connections to the cytoskeleton (FIG. 5B, 5C, 5E), and appear at a lower frequency (per □m) than in wild-type skin. In addition, some desmosomes in Perp null skin exhibit severe defects in their ability to function as stable cell-cell junctions: the desmosome remains intact and attached to one cell with only a fragment of the apposing cell membrane in evidence (FIG. 5C, 5E). This may represent a defect in attachment of the plaque to the intermediate filament network. These abnormalities are quantified in FIG. 5G, and were observed in multiple Perp null mice (n=4) in comparison to wild-type littermates (n=2). Similar defects were also observed in the upper suprabasal layer of the skin (data not shown), and in Perp null tongue epithelium (n=4 mice, FIG. 5F), suggesting common underlying causes for the blistering in both tissues. In contrast, examination of adherens junctions in Perp−/− skin failed to reveal obvious aberrations (data not shown). These data suggest that, while some desmosomes are able to form in the absence of Perp, the desmosomal proteins do not associate as tightly with each other as they do normally and are defective in their ability to form stable attachments to the cytoskeleton. Because the desmosomes in Perp null skin are compromised in their strength and organization, epithelia in these mice are greatly susceptible to damage after mechanical stress.

Desmosomal Complexes are Defective in the Absence of Perp. The compromised desmosome function in Perp null animals could be explained by deficiencies in levels, localization, or plasma membrane organization of desmosomal constituents. Proteins stably incorporated into the desmosome are characterized by their resistance to solubilization by detergents such as Triton X-100, and therefore increased solubility of desmosomal cadherins or plaque proteins often reflects improper complex assembly (South et al., 2003). However, desmoplakin, which links the desmosomal plaque to the keratin cytoskeleton, is known to exist in Triton X-100-insoluble cytoplasmic bodies prior to desmosome assembly (Green and Gaudry, 2000), and its properties cannot be assessed clearly by solubility assays. We therefore employed both immunofluorescence and biochemical assays to investigate the cause of the desmosome defects observed in Perp−/− skin by EM.

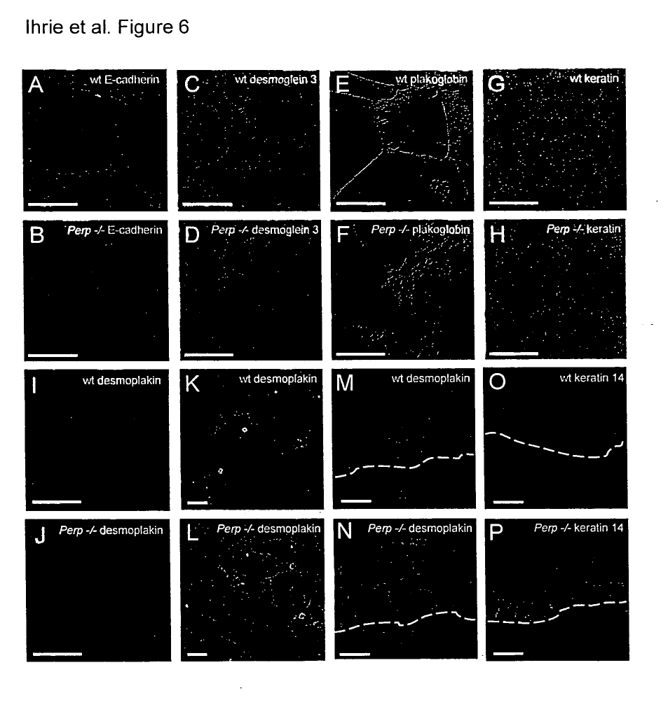

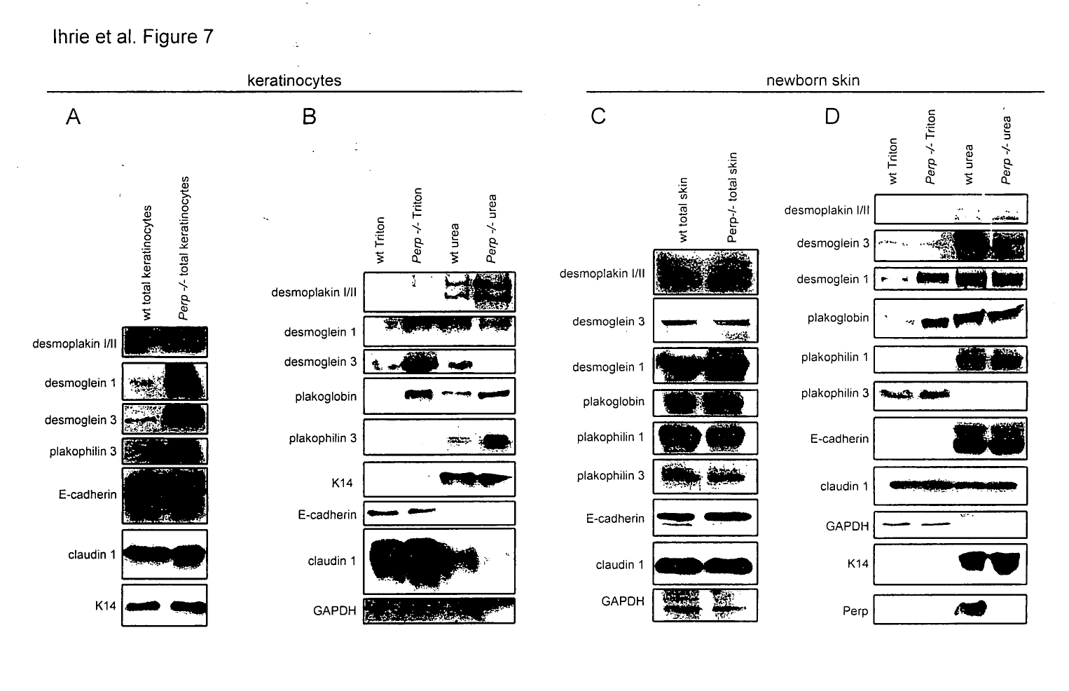

We began our analysis of desmosomal components by examining desmosome protein localization in a cell culture model of adhesion. Primary keratinocytes can be induced to form epithelial sheets in culture through a switch from low to high calcium-containing media (Vasioukhin et al., 2001). After 48 hours in high calcium medium, keratinocytes express high levels of desmosomal proteins which complex at the plasma membrane in the form of mature desmosomes. We derived epidermal keratinocytes from P0.5 wild-type and Perp−/− mice and examined the subcellular localization of desmosomal proteins after induction of desmosome formation (FIG. 6). Although Perp−/− keratinocytes exhibit increased levels of multiple desmosomal proteins (FIG. 7A, 7B), the localization of most desmosomal proteins, including desmoglein 3 and plakoglobin, was largely unaltered in Perp−/− keratinocytes (FIG. 6C-F). In contrast, the staining pattern for desmoplakin appeared different from that seen in wild-type cells (FIG. 6I-L). Whereas most desmoplakin localized to the cell membrane in adherent wild-type keratinocytes, desmoplakin exhibited diffuse intracellular staining with minimal plasma membrane targeting in Perp−/− keratinocytes.

Although the increase in cytoplasmic desmoplakin could result from the higher protein levels present in Perp−/− cells, the fact that other overexpressed desmosomal components target appropriately to the plasma membrane supports the idea that there may be a specific defect in desmoplakin localization. Consistent with our results in this keratinocyte model, we found that in skin from wild-type and Perp−/− newborn mice, desmoglein 3 and plakoglobin localized primarily to the cell periphery while desmoplakin showed some cytoplasmic retention and keratin 14 aggregated abnormally within the cell (FIG. S5C-F, 6M-P). In the skin, desmoplakin levels are not affected by Perp-deficiency, further suggesting that this is not the only basis for the mis-localization observed in keratinocytes. Finally, examination of E-cadherin and actin staining revealed that formation of adherens junctions in Perp−/− keratinocytes and skin appears unaltered, underscoring the specific effect of Perp loss on desmosomes (FIG. 6A, 6B, S5A, S5B.

We next determined the relative amounts and solubility profiles of desmosomal components in wild-type and Perp−/− skin. Examination of total desmosomal protein levels demonstrated that there is no significant alteration of protein levels in Perp−/− skin relative to wild-type skin (FIG. 7C). We then determined which proteins could be extracted by Triton X-100 and which could be solubilized only by chaotropic agents, the classic behavior for assembled desmosomal proteins (Bornslaeger et al., 2001; Vasioukhin et al., 2001). Interestingly, we observed that Perp partitioned into the urea fraction, similar to other desmosome-associated proteins, supporting the idea that Perp is a desmosomal constituent (FIG. 7D). Furthermore, we found that some desmosome proteins, specifically desmoglein 1 and plakoglobin, are greatly enriched, and desmoglein.17 3 levels are slightly enhanced, in the Triton X-100-soluble fraction from Perp−/− skin relative to wild-type skin (FIG. 7D). Although total protein levels of desmosomal constituents were higher in cultured keratinocytes from Perp−/− mice than those from wild-type mice, suggesting adaptation to culture conditions in the absence of Perp, the pattern of altered solubility was similar to that seen in skin (FIG. 7A, 7B). Together, these findings indicate that the fraction of proteins efficiently incorporated into the desmosome is diminished in Perp−/− epithelia, supporting a role for Perp in desmosome assembly or stability. Such a defect also explains the less electron-dense appearance and increased width of desmosomes detected in Perp−/− skin compared to wild-type skin by EM. These solubility effects appear to be specific to desmosomes, as the adherens junction and tight junction components E-cadherin and claudin 1 displayed unaltered properties in Perp−/− skin and keratinocytes (FIG. 7A-D).

These data collectively support a role for Perp in proper desmosome function. As desmoplakin is required to link the desmosomal plaque to the keratin intermediate filament cytoskeleton, any abnormal cytoplasmic accumulation in Perp−/− cells suggests a possible rationale for the lack of attachment of desmosomes to the cytoskeleton observed by EM. Additionally, although plakoglobin and the desmogleins apparently target to the plasma membrane, their increased solubility in Perp−/− cells and skin relative to wild-type controls suggests that despite proper localization, they may not be correctly incorporated into desmosomes. The decreased stability of desmosomal complexes provides a basis for the observed malfunction of desmosomes in Perp−/− epithelia and underscores the central role for Perp in desmosome function and epithelial integrity.

Perp is a p63-Regulated Gene. Here, through analysis of knockout mice lacking the p53 apoptosis-associated target gene Perp, we reveal a novel and fundamental role for Perp in promoting cell-cell adhesion and maintaining epithelial integrity. The phenotype of Perp−/− mice, which display severe blisters in tissues prone to stress as well as postnatal lethality, contrasts with the viability of p53−/− mice, indicating a p53-independent developmental function for Perp. This result, along with several other findings, implicates Perp instead in a pathway downstream of the p53-related protein p63 in the process of stratified epithelial development. First, the observation that p63 and Perp have very similar expression profiles in E9.5-10.5 embryos, and in a variety of stratified epithelia in older animals, initially suggested that Perp may be directly regulated by p63. Second, like Perp, p63 plays a crucial part in the establishment of stratified epithelia, including the skin and oral mucosa, although the role of p63 is more global and the role for Perp more specialized. p63 is necessary for the genesis of stratified epithelia, as demonstrated by the absence of these structures in p63−/− mice (Mills et al., 1999; Yang et al., 1999). Moreover, expression of p63 is sufficient to induce simple epithelia to express markers of stratified epithelia, illustrating that p63 acts as a master switch directing the initiation of epithelial stratification. As part of this process, p63-mediated induction of a transcriptional program specifying stratified epithelial development likely includes activation of a set of genes involved in epithelial specialization, such as Perp.

Consistent with the notion of a p63-Perp pathway, we have demonstrated here that Perp expression in the developing embryo and in cultured keratinocytes depends on.19 p63. That p63 directly induces Perp is demonstrated by reporter assays and chromatin immunoprecipitation experiments showing p63 binding to the Perp regulatory region in vivo. Furthermore, we show that all p63 isoforms examined, including both TA and .N isoforms, are competent to activate expression of the Perp luciferase reporter. Although the exact roles of the multiple p63 isoforms in vivo are incompletely understood, it is proposed that they have distinct roles during skin development, with TAp63 and ΔNp63 playing roles in commitment to stratification during embryogenesis and epidermal differentiation in mature skin, respectively (McKeon, 2004; Koster et al, 2004). Our data suggest that Perp is regulated by more than one p63 isoform in the skin, which may reflect a broad requirement for Perp at different times during stratified epithelial development as well as in all layers of the tissues specified by p63. The prevailing notion is that ΔNp63 isoforms act primarily to inhibit p53 or p63 target gene activation. Our results, along with other recent studies (Ellisen et al, 2002), indicate that the scenario is more complex than originally appreciated: ΔNp63 is not simply a negative regulator, but retains activity to stimulate expression of certain genes. This suggests that there may be different classes of p63 target genes that respond differentially to the various isoforms of p63, as a reflection of their biological function. Perp is a target gene that can be activated by all isoforms of p63, and is induced at multiple stages of development, consistent with the requirement for proper adhesion throughout development. Thus, Perp is the first p63 target with a demonstrated functional role consistent with regulation by both sets of isoforms, including the ΔNp63 isoforms thought to predominate in the mature skin.

These studies identify Perp induction as a critical sub-program in the specification of stratified epithelia by p63. The mode by which p63 specifies skin development is controversial, with roles either in stem cell maintenance or in commitment to stratification being proposed. Our findings support a model in which p63, during induction of a stratification program, transactivates a cohort of genes that endow a stratified epithelium with specific characteristics rather than simply maintaining the epidermal proliferative compartment.

Perp Plays a Central Role in Stratified Epithelial Integrity by Promoting Desmosome Assembly. Perp's specific role within the framework of the p63 developmental program for stratified epithelia is in establishing cell-cell adhesive contacts. In particular, Perp localizes to desmosomes and is required for proper desmosome formation in stratified epithelia, as demonstrated by the abnormal morphology of desmosomes and the altered properties of desmosomal components in Perp−/− skin. Given the association of compromised desmosome function with various human skin disorders, these results suggest the tantalizing possibility that Perp mutations may be implicated in human blistering diseases of unknown etiology. Two general models may explain how Perp might participate in desmosome assembly and function. Perp's contribution to desmosomal integrity could be as a core structural component, or alternately, as a chaperone that facilitates the transit of other critical desmosome components to the plasma membrane. Some desmosomal constituents, such as the transmembrane desmosomal cadherin molecules, which engage in heterotypic interactions between neighboring cells and nucleate the desmosomal plaque protein complex at the cytoplasmic face of the cell, are clearly involved in establishing the architecture of the desmosome. Likewise, Perp could participate either in homotypic or heterotypic interactions at the plasma membrane to provide important adhesive contacts that constitute part of the desmosomal framework. Another potential structural role for Perp is as an anchoring point for connections,to the intermediate filament cytoskeleton.

As a chaperone, Perp might assist in the trafficking or assembly of desmosomal subunits. In the case of adherens junction complexes, β-catenin acts as a molecular chauffeur for E-cadherin, facilitating its shuttling from the secretory pathway to the plasma membrane. Examination of the migration of desmosome components to the plasma membrane has suggested that the desmosomal cadherins and desmoplakin transit to the plasma membrane through two different compartments, and assemble to form the desmosome after reaching the membrane. In particular, desmoplakin has been described to localize to discrete packets in the cytosol, and to travel to the cell surface via the intermediate filament network upon receiving the proper cue. Plakophilin 1 participates in the recruitment of desmoplakin to the plasma membrane as skin cells from patients with Plakophilin 1 mutations display cytoplasmic desmoplakin localization and defects in desmosomal plaque-intermediate filament connections. However, other facets of this assembly and trafficking process are as yet uncharacterized, leaving open the possibility that Perp may participate in these events. There is precedent for tetraspan proteins acting as molecular escorts or organizing factors for membrane proteins. For example, stargazin, a member of the claudin/PMP-22/ EMP family, is implicated in the delivery of the AMPA receptor to the plasma membrane of cerebellar granular neurons, as well as in the clustering of these receptors at the synapse. This scenario is likely to be paradigmatic for tetraspan membrane proteins, in which they actively promote transiting, organization or stabilization of plasma membrane proteins into complexes essential for effecting specific cellular processes. Perp may play a role in the shuttling, assembly or stabilization of desmosomal proteins.

Perp is Important for Tissue Homeostasis Downstream of Both p63 and p53. Our findings demonstrate a connection between Perp's roles in apoptosis and adhesion. While the mechanism by which Perp participates in p53-mediated apoptosis is not well understood, its activities in programmed cell death and adhesion may share common features. Perp activity in each of these processes may reflect a shared cell biological role that is relevant to both cellular responses, or alternatively, a signaling role that differs according to developmental context. A general function for Perp could relate to the shuttling or assembly of membrane proteins at the plasma membrane: death receptors in the case of apoptosis, and adhesion components in the case of desmosomes. Alternatively, Perp may utilize distinct activities to enable the apoptotic and adhesion responses.

In its roles downstream of both p53 and p63, Perp is implicated in signaling important for tissue integrity. The p53 tumor suppressor is known as a “guardian of the tissue”, with an essential function in maintaining tissue homeostasis. In response to cellular stresses including DNA damage or hyperproliferative signals, p53 induces apoptosis, in part through Perp, as a measure to protect an organism against malignancy. Similarly, the p63-Perp axis may be involved in the establishment or maintenance of tissue integrity, in this case by facilitating appropriate cell-cell contacts necessary for tissue function. The p53 and p63 pathways may intersect to integrate signals that control tissue homeostasis in different settings, in some cases through apoptosis, in some cases through adhesion. We propose signaling complexes that sense the local environment and thereby promote either apoptosis or adhesion, with Perp playing a key role in both processes.

Experimental Procedures

In Situ Hybridization. In situ hybridization with digoxigenin- or ³⁵S-radiolabeled probes was carried out as described, using a probe transcribed from a plasmid containing the full-length Perp cDNA (pT7T3Pac-mPerp) (Frantz et al., 1994; Yang et al., 1999). For section in situ hybridization, 6 μm frozen sections from E16.5 embryos were used.

Northern Blot Analysis. RNA samples were isolated from snap-frozen tissue or cultured cells using Trizol (Invitrogen). Northern blotting was performed according to standard methods.

Retroviral infection of primary keratinocytes Retroviruses were produced by transiently transfecting Phoenix-E cells with constructs expressing shRNA targeting exon 4 of p63 (p63 hairpin 1, or D8, anti-sense target sequence: GACTGCTGGAAGGACACATCGAAGCTGTG; pMSCVpuro-ZZ-SHAG-LICaka4A) or exon 6 of p63 (p63 hairpin 2, or D9, anti-sense target sequence: GTGATAGGATCTTCTACATACTGGGCATG) or with a GFP-expressing control (pMSCV-puro-PIG; Hemann et al., 2003), as described. Infections were performed as described (Hemann et al., 2003), and keratinocytes were cultured for 72-96 hours prior to RNA and protein harvesting.

Reporter assays 50,000 p53−/−,p63−/− MEFs or primary human keratinocytes were plated in wells of a 24-well plate. The next day, cells were transiently transfected using Fugene 6 (Roche) with 250 ng of empty vector (pcDNA3.1), a p53 expression vector (pCMVp53WT; Reczek et al., 2003), or vectors encoding the indicated p63 isoforms (Yang et al., 1998) and 250 ng of either the pPerpLucPS reporter construct or pPerpLucPSmutD (Reczek et al., 2003) as well as 25 ng of pRLnull (Promega). After 24 hours, cells were harvested and luciferase activity was measured using the Dual-Luciferase Reporter Assay system (Promega). Firefly luciferase values were divided by Renilla luciferase values to control for transfection efficiency. Each experiment was performed at least 3 times, with duplicate samples in each experiment.

Chromatin Immunoprecipitation. Chromatin was prepared by isolating P1.5 skin and fixing immediately in formaldehyde for 10 minutes. A single-cell suspension was made from fixed skin, cells and nuclei were lysed, and chromatin was sonicated to an average length of 800 bp before incubation with the 4A4 anti-p63 antibody (Santa Cruz) or an isotype control nonspecific antibody. After immunoprecipitation with Protein A/Protein G beads (Pharmacia), crosslinks were reversed and isolated DNA was used for PCR with primers flanking the Perp p53/p63 consensus site D or primers for an irrelevant region of Perp not containing a consensus site.

Adenoviral infection p63−/− cultured El 8.5 ectoderm cells were plated for 48 hours after isolation. Cells were infected with Ad-pShuttle-CMV-TAp63 or Ad-pShuttle-CMV (Ellisen et al., 2002), for 2 hours. Cells were harvested for protein and RNA at 24 and 36 hours after infection, respectively.

Histology and Immunohistochemistry Paraffin sections from newborn and P7.5 mice were prepared for immunohistochemical or hematoxylin/eosin staining by standard methods. Immunohistochemistry was performed according to standard methods, with permeabilization for antigen retrieval by microwaving in 0.01 M citrate buffer (pH 6.0) or 1M Tris (pH 9.5),.5% urea buffer.

EM and Image Analysis For immunogold labeling, ventral skin sections were taken from P0.5 mice and embedded in Lowicryl K4M resin at −35 C, sectioned, and mounted on nickel grids before labeling with anti-Perp antibodies and colloidal gold-conjugated secondary antibodies. For transmission EM, pieces of tongue and ventral skin were taken from P0.5 newborn mice and prepared by standard methods. Sections were imaged on a JEOL TEM 1230 microscope with a Gatan 967 CCD camera (Stanford Electron Microscopy Core).

Desmosome properties in 50000X magnification EM images were quantified as follows: Total continuous membrane length and individual desmosome length along the plasma membrane were measured and normalized against the scale bar within each image. Individual desmosomes were scored for electron density as light (plasma membrane visible) or dark (not possible to see membrane), attachment to intermediate filaments, and as normal or abnormal based on the above characteristics or detachment from apposed cells. Measurements were compiled for statistical analysis using GraphPad Prism.

Protein Preparation and Immunoblotting For skin protein preparation, skin was snap-frozen and homogenized using a chilled mortar and pestle. For keratinocyte protein preparation, plated cells were washed with PBS and chilled buffer was added. For Triton-soluble protein fractions, cells or skin samples were resuspended in 1% Triton X-100 solubilization buffer (Vasioukhin et al., 2001), rocked for one hour at 4° C., and the supernatant was isolated by centrifugation. The urea-soluble protein fraction was obtained by resuspending the Triton-insoluble material from this preparation in the same buffer + 9M urea. Total protein extracts were made by direct lysis in 9M urea buffer. Western blotting was performed according to standard methods, with 25-50 μg of protein in each lane.

Keratinocyte Culture and Adhesion Assays E18.5p63−/− ectoderm or skins from P0.5 mice were prepared as described (Koster et al., 2004), pooled by genotype and plated in DMEM containing 1.2 mM calcium, fetal calf serum (FCS), and growth factor supplements. For calcium-dependent adhesion assays, cells were washed 3× with Ca₂₊-free PBS the next day and re-fed with EMEM containing 8% dialyzed FCS, antibiotics, and 0.05 mM Ca₂₊. Cells were grown to ˜80% confluency, washed with PBS, and incubated in the same media with 1.2. mM Ca₂₊ to induce epithelial sheet formation. After 48 hours, cells were harvested for protein extracts or immunofluorescence.

Immunofluorescence Keratinocytes cultured on 35 mm plates with sterile glass coverslips were fixed in either cold 2% paraformaldehyde in PBS for 20 minutes or in chilled methanol for 5 minutes. Samples of skin from newborn mice were prepared by embedding the specimens in OCT. 5-7 ∝m frozen sections were fixed 5 minutes in 1:1 methanol:acetone and allowed to air dry. Immunofluorescence was done as described (Attardi et al., 2000).

Antibodies For immunofluorescence and immunohistochemistry, we used antibodies against Ki67 (Pharmingen), desmoplakin I/II (RDI), desmoglein 1 (4B2, gift of K. Green, Northwestern), desmoglein 3 (gift of J. Stanley, U. Pennsylvania), E-cadherin (Zymed), plakoglobin (1407, gift of K. Green, Northwestern), ZO-1 (gift of W. J. Nelson, Stanford), plakophilin 1 (RDI), keratins 14, 1, and 6 and Ioricrin (Covance). For immunoblotting, we also used antibodies against p63 (4A4, Santa Cruz), desmoglein 3 (Santa Cruz), claudin 1 and plakophilin 3 (Zymed), and GAPDH (RDI). Polyclonal antibodies against Perp were generated by injection of a carboxy-terminal peptide fragment ((C)NYEDDLLGAAKPRYFY, Zymed), and affinity purified by coupling the peptide to Sulfolink resin (Pierce) following the manufacturer's directions.

### Example 2

In its capacity as a p63 target, Perp functions in specific cell-cell adhesion complexes known as desmosomes. Desmosomes are essential both for anchoring cells to each other and for conferring strength on a tissue by virtue of contacts to the intermediate filament cytoskeleton, a facet especially important in tissues subject to mechanical stress. Perp localizes to desmosomes, and cells in the epidermis of Perp-deficient mice display aberrant assembly of desmosomal complexes both by biochemical assays and electron microscopy, indicating the central role for Perp in desmosomal function. Thus Perp is a key effector of the p63 stratified epithelial development program, mediating a sub-program specifically involved in cell-cell adhesion.

Given that Perp is positioned downstream of both p53 and p63, and participates in both apoptosis and adhesion, loss of Perp may be expected to promote cancer. To test potential tumor suppressor activity of Perp, we utilized a carcinogenesis model in the skin, a tissue where Perp is known to play an important role. Here, by examining conditional knockout mice in which Perp is ablated in the skin, we define the role for Perp in skin tumorigenesis. Surprisingly, we find that Perp-deficient mice are resistant to cancer, suggesting that intact Perp function is required for efficient tumorigenesis.

Materials and Methods

Skin carcinogenesis protocol. Male K5-Cre; Perp fl/+ were mated to Perp fl/+ females to generate mice used for this study, including 17 K5-Cre; Perp+/+ mice, 12 K5-Cre; Perp fl/fl mice, and 11 K5-Cre Perp fl/+ mice. Mice were of a mixed 129/Sv/C57BL/6 genetic background and littermates were utilized for experiments. Each mouse was genotyped by PCR. To detect the Cre transgene, we used the primers 5′-TGG GCG GCA TGG TGC AAG TT-3′ and 5′-CGG TGC TAA CCA GCG TTT TC-3′. To determine the Perp genotype, we used primers located in the first intron, 5′-AGT CTT CAG GGA TGA CAC AGA and 5′-TAC GAA ACT AGA GCA CAG CTA-3′, which result in a 326 bp product on the wild-type allele and a 410 bp product on the conditional allele.

The backs of 8 week-old mice were shaved and treated with a single application of 7,12-dimethylbenz(a)anthracene (DMBA, Sigma; 10 μg in 100 μl acetone) followed by twice weekly application of 12-O-tetradecanoylphorbol 13-acetate (TPA, Sigma; 12.5 μg in 100 μl acetone) for 27 weeks. The numbers and sizes of papillomas were recorded once a week. With the exception of 2 K5-Cre; Perp+/+ mice and 6 K5-Cre; Perp f mice that died between week 24 and 28, mice were sacrificed 27 weeks after the initiation of TPA treatment (week 28), and skin with and without papillomas was fixed in 10% buffered formalin.

Histology and Immunohistochemistry. Paraffin sections from adult mouse skin were prepared for immunohistochemical or hematoxylin and eosin staining by standard methods. Immunohistochemistry was performed as previously described.

TUNEL and Ki67 Assays. TUNEL assays were performed as described (13). Ki67 antibody labeling was detected using a Mouse-on-Mouse Kit with biotin-labeled secondary (Vector) and Cy3-streptavidin (Jackson ImmunoResearch). Proliferation was quantified by counting the number of labeled cells as a percentage of total basal cells in each 20× field. Assays were performed on treated skin from mice at the end of the study.

Protein Preparation and Immunoblotting. For skin protein preparation, skin was snap-frozen in liquid nitrogen, and homogenized using a chilled mortar and pestle. Triton-soluble and urea-soluble protein fractions were prepared as previously described. Western blotting was performed according to standard methods, with 25 μg of protein per lane.

Antibodies. For immunofluorescence and immunohistochemistry, antibodies against Ki67 (Pharmingen) as well as keratin 14, keratin 1, and Ioricrin (Covance) were utilized. For immunoblotting, antibodies against desmoglein 1 (4B2), plakoglobin (1407), and tubulin (Sigma) were used. Polyclonal antibodies against Perp are described above.

Results

Specific inactivation of Perp in Skin. As constitutive Perp-deficient mice display post-natal lethality, we took advantage of Perp conditional knockout mice that we generated to establish the role of Perp in skin carcinogenesis (FIG. 8A). To ablate Perp in the skin, we bred conditional, or floxed, Perp mice (denoted Perp fl/fl) to Keratin 5-Cre transgenic mice expressing Cre specifically in stratified epithelia, including the skin. We verified stratified epithelia-specific expression of Cre in these transgenic mice by crossing them to a Z/AP reporter strain. We detected robust reporter expression in the skin of K5-Cre; Z/AP compound mice, confirming that this would serve as an ideal model system for examining Perp function in the skin. Some of the compound K5-Cre; Perp fl/fl mice died within 10 days after birth, similar to the partially penetrant lethality observed in Perp null newborns. Examination of tissues from adult survivors by western blot analysis verified that Perp protein is indeed absent in stratified epithelia from K5-Cre; Perp fl/fl mice compared to mice lacking the transgene (FIG. 8B). To confirm the efficiency of Perp deletion in the skin, we examined Perp staining by immunohistochemistry.

Whereas Perp plasma membrane staining was observed in K5-Cre; Perp+/+ control adult mice, Perp staining was undetectable in the K5-Cre; Perp fl/fl adult mice (FIG. 8C, D). These findings indicate that the surviving K5-Cre; Perp fl/fl adults provide a useful model in which to examine the effect of Perp-deficiency on skin cancer development.

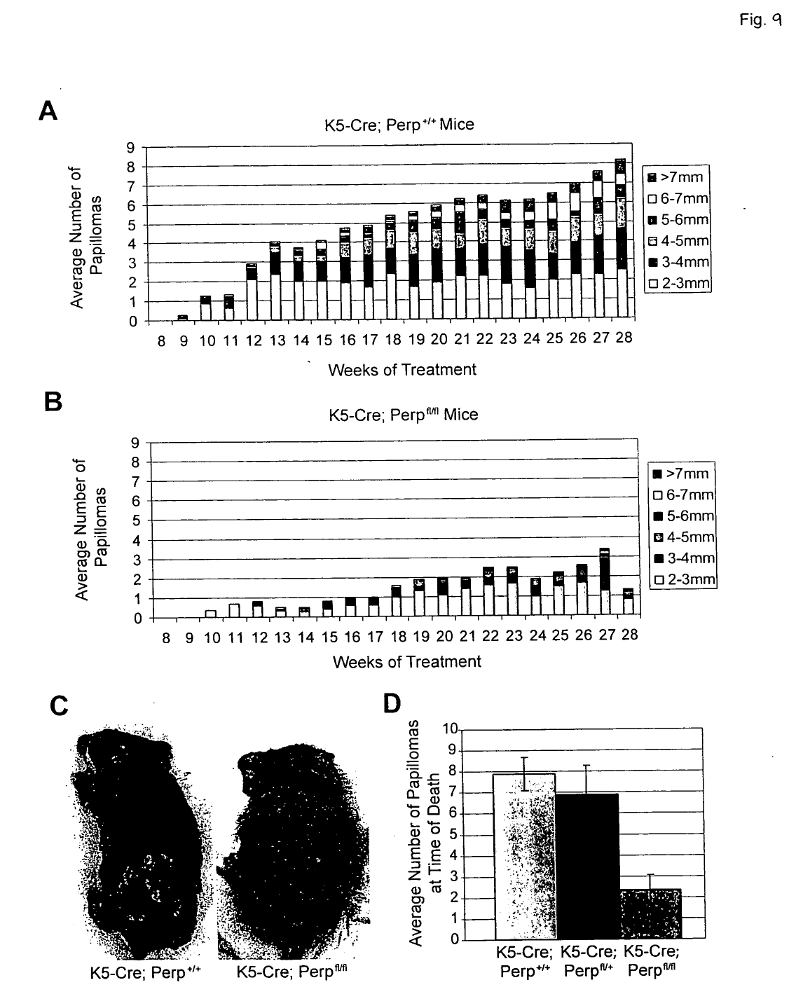

Perp-deficient mice are resistant to skin papillomagenesis. To determine the role of Perp in skin papilloma development, we subjected cohorts of K5-Cre; Perp fl/fl and K5-Cre; Perp +/+ control adult mice to a well-established two-step skin carcinogenesis protocol. In this protocol, tumor initiation results from application of the carcinogen DMBA, which induces activating ras mutations, and tumor promotion ensues after multiple weeks of treatment with the phorbol ester TPA. Mice were exposed to one treatment with DMBA followed by 27 weeks of bi-weekly TPA treatment, and both numbers and sizes of developing papillomas were monitored during this period. Papillomas began to appear in both groups at about the same time, approximately 8-9 weeks after treatment initiation. In K5-Cre; Perp+/+ mice, the numerous papillomas that appeared grew progressively larger throughout the treatment period (FIG. 9A, C).

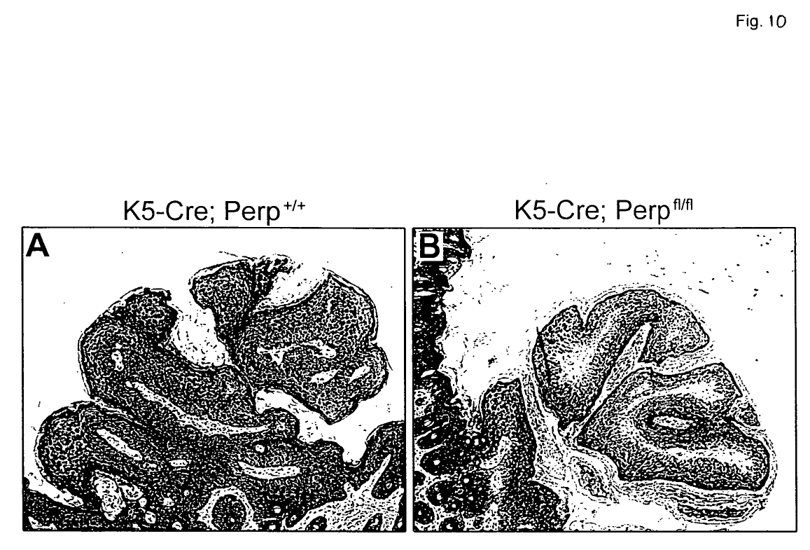

In striking contrast, K5-Cre; Perp fl/fl mice were highly resistant to papillomagenesis, typically displaying only a minuscule number of tumors, that were diminished in size (FIG. 9B, C). At the time of death, the average number of papillomas in the Perp-deficient mice was approximately 4-fold less than the wild-type mice (FIG. 9D). The difference in papilloma numbers, as well as size, between the wild-type and the Perp-deficient mice at the end of the study suggests that Perp is important for efficient papilloma development (FIG. 9A-D). Interestingly, mice heterozygous for the Perp conditional allele developed the same number and size of papillomas as wild-type mice, indicating that one copy of Perp is sufficient to facilitate papilloma development (FIG. 9D). However, except for the size differences, the gross histological appearance of the papillomas was not obviously different between the K5-Cre; Perp +/+ and K5-Cre; Perp mi mice (FIG. 10). In addition, none of the papillomas in these cohorts of mice progressed to malignant carcinomas. As our mice are on a mixed 129/Sv;C57BL/6 background, this may relate to the previously described resistance of C57BL/6 mice to malignant conversion in this model system.

Perp-deficient skin displays normal proliferation, apoptosis and differentiation profiles. The dramatic inhibition in papilloma development observed in the K5-Cre; Perp fl/fl mice relative to K5-Cre; Perp+/+ mice could be accounted for by decreased proliferation or enhanced apoptosis in the absence of Perp. To determine if there were inherent differences in the properties of keratinocytes in the wild-type and Perp-deficient mice that could result in differences in the propensity to papillomagenesis, we examined levels of cell division and apoptosis in treated skin from mice of both genotypes.

To establish proliferation indices, we measured the percentages of Ki67 positive basal cells in the skin of treated Perp-deficient and wild-type mice (FIG. 11A-D). We found that the fraction of Ki67 positive cells in the Perp-deficient skin was indistinguishable from that observed in wild-type skin, indicating that a difference in the proliferative capacity of the cells of the treated skin does not account for the relative resistance of Perp-deficient mice to skin carcinogenesis (FIG. 11E). In addition, no clear difference in proliferation levels was observed in the developing papillomas of K5-Cre; Perp fl/fl mice relative to K5-Cre; Perp+/+ mice.

Given Perp's established role in apoptosis, we sought to determine if apoptosis levels were altered in the absence of Perp. Terminal deoxynucleotidyl transferase dUTP Nick End Labeling (TUNEL) staining was performed on K5-Cre; Perp+/+ and K5-Cre; Perp fl/fl mouse skin (FIG. 11F, G). TUNEL staining failed to reveal significant numbers of apoptotic cells in treated skin derived from mice of either genotype. Moreover, apoptotic levels were also minimal in papillomas derived from both cohorts of mice. These findings indicate that augmented apoptosis does not explain the inability of papillomas to form efficiently in the absence of Perp.

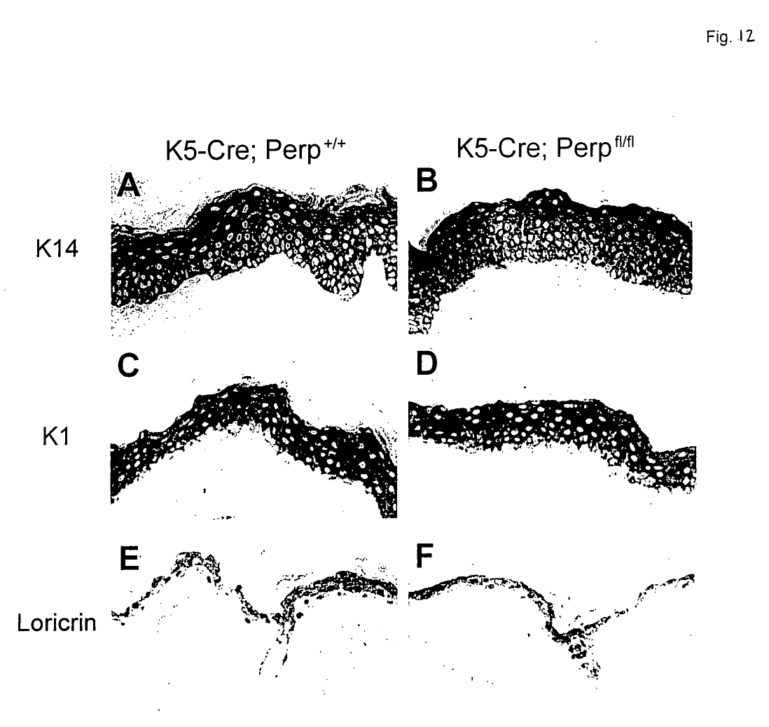

One additional possibility is that papillomagenesis is impeded in the absence of Perp because of aberrant differentiation of keratinocytes in the skin, as has been observed with certain mouse models examined in the two-step protocol. To address this issue, the differentiation program in treated skin from K5-Cre; Perp+/+ and K5-Cre; Perp fl/fl mice was examined (FIG. 12).

Staining for K14, K1 and Ioricrin—markers of the basal, spinous and granular layers of the epidermis, respectively—was performed. All of these markers were detected irrespective of Perp genotype, indicating that the differentiation process occurs normally, and suggesting that the disruption of differentiation is not the basis for the inhibited papillomagenesis in K5-Cre; Perp fl/fl mice (FIG. 12). Taken together, these findings suggest that neither the cell number nor the differentiation state in the treated skin vary with Perp status, but rather some other property of the skin may differ. Given the previously described role for Perp as a crucial desmosomal protein, we hypothesized that adhesion differences might explain the differences in papilloma development observed in the two cohorts.

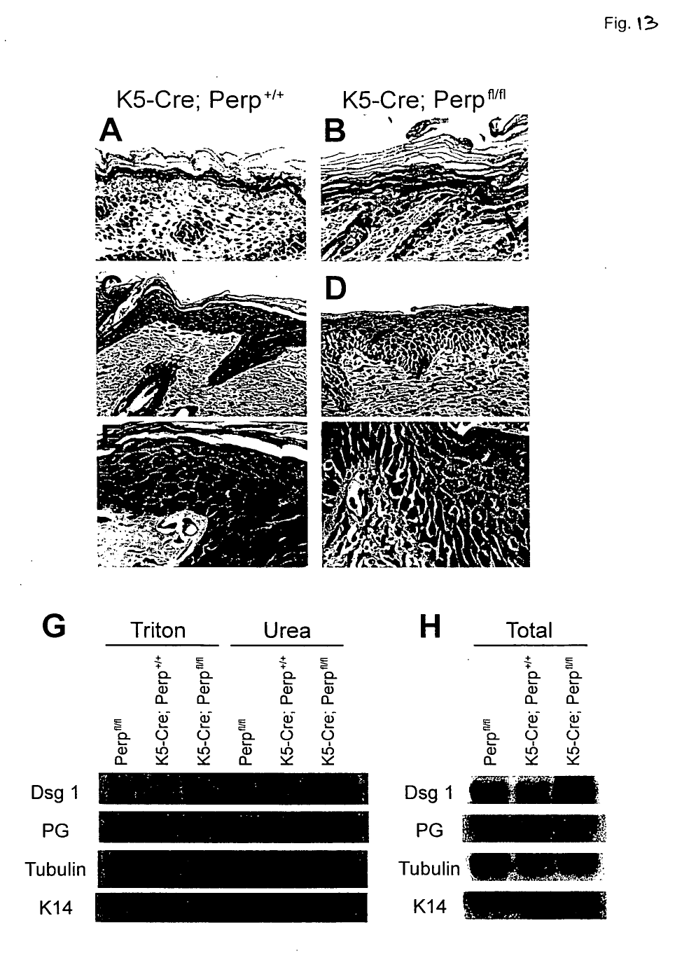

Significant Adhesion Defects Are Observed in Perp-Deficient Skin. Perp−/− mice are prone to the development of blisters, or separation between epithelial layers, as well as separation between individual epithelial cells in the skin due to disrupted desmosome function. To test the hypothesis that compromised desmosomal function may form the basis for the decreased susceptibility to papilloma development in. K5-Cre; Perp fl/fl mice, we examined adhesion in the skin of these mice. Skin from untreated newborn K5-Cre; Perp fl/fl mice showed a propensity to develop blisters, similar to that observed in Perp constitutive null animals, indicating that clear adhesion defects are observed when Perp is deleted selectively in the skin (FIG. 13A, B). To specifically determine if adhesion is disrupted in the adult mice subjected to the carcinogen protocol, we examined the treated skin by histological analysis. Interestingly, while the keratinocytes in the K5-Cre; Perp+/+ mice showed normal attachment to each other, cells in the K5-Cre; Perp fl/fl mouse skin displayed clear signs of separation (FIG. 13C-F, arrows). These results suggest that adhesion is impaired in the skin of Perp-deficient mice.

To show specifically that desmosome function is responsible for the observed alterations in adhesion, we utilized an assay that we established previously as a measure of desmosomes integrity in studies of Perp−/− mice (14). When desmosomes undergo proper assembly, desmosomal components are largely insoluble in the detergent Triton X-100. If the assembly or stability of the desmosome is abnormal, however, then desmosomal proteins can be solubilized by Triton X-100 to a greater extent than usual. In skin from newborn Perp−/− mice, the desmosomal proteins desmoglein 1 and plakoglobin display increased solubility relative to skin from wild-type mice. This biochemical property is accompanied by clear defects in desmosomes structure at the electron microscopy level (14, 24).

We analyzed the solubility of these central desmosomal proteins in untreated skin of K5-Cre; Perp+/+ mice and K5-Cre; Perp fl/fl adult mice. Both desmosomal constituents examined, desmoglein 1 and plakoglobin, were more Triton-X-100-soluble in the K5-Cre; Perp fl/fl mouse skin than in control skin, indicating that, in the absence of Perp in the skin, normal assembly of mature desmosomes is compromised (FIG. 13G), consistent with our previous observations in the skin of newborn constitutive Perp−/− mice. Total levels of these desmosome proteins do not vary, indicating that the observed effects relate specifically to solubility differences (FIG. 13H). Together, our results demonstrate that in the absence of Perp, skin tumorigenesis is impeded, and that proper desmosome function is important for tumorigenesis.

Here, we test the role of the p53/p63 target gene Perp in cancer development using a two-step skin carcinogenesis model. Based on the central roles for Perp in both apoptosis and adhesion, we hypothesized that Perp-deficient mice would display an enhanced predisposition to cancer. Surprisingly, however, we observed a striking resistance to tumorigenesis in mice lacking Perp in the skin, with diminished numbers and sizes of skin papillomas. In a variety of mouse models, similar resistance to papilloma development is clearly correlated with inherent changes in proliferation, apoptosis, or differentiation. In contrast, in the treated skin of mice lacking Perp, we observed no such alterations. Instead, we observed defects in adhesion, specifically in desmosome assembly, in the skin of Perp-deficient mice. Our findings indicate that Perp is important for skin tumorigenesis in this system, and suggest a requirement for intact desmosome function during this process.

Given Perp's central function in adhesion in the skin, it is likely that its ability to facilitate skin papilloma development is related to this activity. It is well-established that the development of cancer requires the dysregulation of adhesion. In particular, changes in adhesion have been demonstrated to play an important part in later stages of cancer, where disrupted cell-cell contacts contribute to the processes of invasion and metastasis. Our findings, in contrast, suggest that proper cell-cell adhesion may be required, at least in some contexts, for the establishment and growth of tumors. The resistance of Perp-deficient cells to tumor development may reflect an intrinsic alteration in the behavior of these cells resulting from adhesion defects, such as enhanced motility or altered cell-cell signaling, which may somehow impede tumor development. Alternatively, it may be that efficient tumor morphogenesis and formation of the 3-dimensional papilloma structure relies on appropriate cell-cell adhesive contacts, and when these are perturbed, tumor formation is inhibited.

The importance of external cues, specifically cell-extracellular matrix (ECM) contacts between epithelial cells and the basement membrane, in tumorigenesis has been suggested previously through a similar skin carcinogenesis study in mice lacking Focal Adhesion Kinase. Keratinocytes in Fak± mice have impaired integrin-mediated signaling, which results in an inhibition of papilloma development similar to that observed here. Similarly, expression of α6β4-integrin and its ligand laminin 5 have been shown to be critical for squamous cell tumorigenesis. Together with our study, these results implicate both proper cell-ECM and cell-cell adhesion in supporting tumorigenesis.

Our findings do not exclude the possibility that adhesion through desmosomes may inhibit tumorigenesis in certain instances. In fact, numerous studies have indicated that both desmosomal and adherens junctions components are downregulated during tumorigenesis and that compromised adhesion may promote tumor metastasis. For example, inactivation of the transmembrane component of the adherens junction, E-cadherin, through mutation, promoter methylation, or transcriptional repression, is associated with the progression from adenoma to carcinoma and with the acquisition of metastatic potential. An interesting possibility is that the specific function of adhesion complexes may depend on the particular stage of cancer examined. For example, while the presence of Perp allows early skin carcinogenesis through promoting papilloma development, it may play an anti-cancer role in late-stage cancer by facilitating adhesion, thereby blocking cell detachment and metastasis. This idea is consistent with reports that Perp is downregulated in metastatic compared to primary melanomas. Future experiments will determine if Perp loss promotes tumorigenesis at later stages of carcinogenesis.

The limited papilloma development in Perp-deficient mice is reminiscent of that seen in p53−/− mice, which display a resistance to developing papillomas compared to wild-type or p53 heterozygous mice. The reason for this paradoxical inhibition of tumorigenesis has never been understood. Because Perp is a target of p53, it is tempting to speculate that the cause of the inhibited papillomagenesis observed in the p53-deficient mouse skin could relate to that seen in Perp-deficient mouse skin. However, Perp expression in stratified epithelia such as skin does not depend on p53, and instead relies on p63. Thus, in the absence of p53, Perp expression remains intact, suggesting that the resistance to tumorigenesis in the p53−/− mice has a different basis. It has not yet been possible to examine p63-deficient mice in this two-step protocol because of their neonatal lethal phenotype. Our data suggest the possibility that p63 loss might also inhibit papilloma development in this model, an idea that potentially can be tested in future studies with conditional p63 knockout mice.

The observation that compromised desmosome function impairs tumorigenesis has therapeutic implications. Treatment of cancers of specific types or at particular stages with anti-desmosome molecules could inhibit tumor development. Perp, as a tetraspan membrane protein exposed at the plasma membrane, represents an accessible target.

### Example 3

### Expression of Perp in Carcinomas

Expression of Perp was tested in a variety of carcinoma samples. The samples were obtained from Stanford University-affiliated laboratories. Controls were normal tissues of the same types as the tumors. In addition staining of placenta samples was performed to ensure that antibodies were not acting non-specifically.

Histology and Immunohistochemistry. Immunohistochemistry was performed according to standard methods, with permeabilization for antigen retrieval by microwaving in 0.01 M citrate buffer (pH 6.0) or 1 M Tris (pH 9.5), 5% urea buffer.

Antibodies For immunofluorescence and immunohistochemistry, we used antibodies against Ki67 (Pharmingen), desmoplakin I/Il (RDI), desmoglein 1 (4B2, gift of K. Green, Northwestern), desmoglein 3 (gift of J. Stanley, U. Pennsylvania), E-cadherin (Zymed), plakoglobin (1407, gift of K. Green, Northwestern), ZO-1 (gift of W. J. Nelson, Stanford), plakophilin 1 (RDI), keratins 14, 1, and 6 and Ioricrin (Covance). For immunoblotting, we also used antibodies against p63 (4A4, Santa Cruz), desmoglein 3 (Santa Cruz), claudin 1 and plakophilin 3 (Zymed), and GAPDH (RDI). Polyclonal antibodies against Perp were generated by injection of a carboxy-terminal peptide fragment ((C)NYEDDLLGAAKPRYFY, Zymed), and affinity purified by coupling the peptide to Sulfolink resin (Pierce) following the manufacturer's directions.

Results:

We have examined Perp protein expression by immunohistochemistry in several different human tumor types represented on human tumor arrays. In the analysis thus far, it was determined that Perp is expressed robustly in certain tumor types but not in corresponding normal tissues. These include lung squamous cell carcinoma and hepatocellular carcinoma. Upregulation was also observed in renal cell carcinoma. In addition, we have detected Perp expression in a number of human breast cancer cell lines. These findings demonstrate the utility of Perp as a diagnostic marker and as a therapeutic target in carcinogenesis.

What is claimed is:

1. A method for the diagnosis of pemphigus, the method comprising:
determining the presence of autoantibodies specific for perp in a patient sample.

2. The method according to claim 1, wherein said patient sample is blood.

3. The method according to claim 1 wherein said patient sample comprises epithelial cells.

4. The method according to claim 1, wherein said determining comprises:
contacting a patient sample with Perp polypeptide;
detecting the presence of a complex formed between auto-antibodies and Perp;
wherein an increase in the presence of said complex, compared to control sample, is indicative of pemphigus.

5. The method according to claim 4, wherein said sample is cell-free.

6. A method for identifying an agent that modulates activity of perp, the method comprising:
combining a candidate biologically active agent with any one of:
(a) a perp polypeptide;
(b) a cell comprising a nucleic acid encoding and expressing Perp (exogenous or endogenous); or
(c) a non-human transgenic animal model for Perp function comprising one of: (i) a knockout of a Perp gene; (ii) an exogenous and stably transmitted mammalian Perp gene sequence; and
determining the effect of said agent on skin disease.

7. A method of treating a skin disorder, said method comprising:
administering a therapeutic amount of a biologically active agent that modulates the activity of Perp in an animal with a skin disorder.

8. The method according to claim 6, wherein said skin disorder is skin cancer.

9. The method according to claim 6, wherein said skin disorder is pemphigus.

10. A method assaying for the presence of carcinoma cells in a sample, the method comprising:
assaying a test sample suspected of containing carcinoma cells chosen from squamous cell carcinoma of the lung and skin; hepatocellular carcinoma, renal cell carcinoma, basal cell carcinoma and breast carcinoma for overexpression of Perp relative to normal tissue of the same type, wherein over-expression of Perp indicates the presence of said carcinoma.
